(19) United States
(12) Patent Application Publication
(10) Pub. No.: US 2008/0242976 A1
(43) Pub. Date: Oct. 2, 2008

(54) Electric field tomography

(75) Inventors: Timothy L. Robertson, Belmont, CA (US); Olivier Colliou, Los Gatos, CA (US); Benedict J. Costello, Berkeley, CA (US); George M. Savage, Portola Valley, CA (US); Todd Thompson, San Jose, CA (US); Mark J. Zdeblick, Portola Valley, CA (US)

Correspondence Address: BOZICEVIC, FIELD & FRANCIS LLP;(PROTEUS BIOMEDICAL, INC), 1900 UNIVERSITY AVENUE, SUITE 200, EAST PALO ALTO, CA, 94303, US

(73) Assignee: Proteus Biomedical, Inc.

(21) Appl. No.: 11/664,468

(22) Filed: Mar. 30, 2007

(51) Int. Cl. A61B 5/04

(52) U.S. Cl. 600/425

(57) ABSTRACT

Methods for evaluating motion of a tissue, such as of a cardiac location, e.g., heart wall, via continuous field tomography are provided. In the subject methods, a continuous field (e.g., an electrical, mechanical, electromechanical, or other field) sensing element is stably associated with the tissue location. A property of the applied continuous field is detected with the sensing element to evaluate movement of the tissue location. Also provided are systems, devices and related compositions for practicing the subject methods. The subject methods and devices find use in a variety of different applications, including cardiac resynchronization therapy.

### CROSS-REFERENCE TO RELATED APPLICATIONS

Pursuant to 35 U.S.C. §119 (e), this application claims priority to the filing date of: U.S. Provisional Patent Application Ser. No. 60/617,618 filed Oct. 8, 2004; U.S. Provisional Patent Application Ser. No. 60/665,145 filed Mar. 25, 2005; U.S. Provisional Patent Application Ser. No. 60/696,321 filed Jun. 30, 2005; and U.S. Provisional Patent Application Ser. No. 60/705,900 filed Aug. 5, 2005; the disclosures of which are herein incorporated by reference.

### INTRODUCTION

### BACKGROUND OF THE INVENTION

In a diverse array of applications, the evaluation of tissue motion is desirable, e.g., for diagnostic or therapeutic purposes. An example of where evaluation of tissue motion is desirable is cardiac resynchronization therapy (CRT), where evaluation of cardiac tissue motion as observed by traditional ultrasound techniques is employed for diagnostic and therapeutic purposes.

CRT is an important new medical intervention for patients suffering from heart failure, e.g., congestive heart failure (CHF). When congestive heart failure occurs, symptoms develop due to the heart's inability to function sufficiently. Congestive heart failure is characterized by gradual decline in cardiac function punctuated by severe exacerbations leading eventually to death. It is estimated that over five million patients in the United States suffer from this malady.

The aim of resynchronization pacing is to induce the interventricular septum and the left ventricular free wall to contract at approximately the same time.

Resynchronization therapy seeks to provide a contraction time sequence that will most effectively produce maximal cardiac output with minimal total energy expenditure by the heart. The optimal timing is calculated by reference to hemodynamic parameters such as dP/dt, the first time-derivative of the pressure waveform in the left ventricle. The dP/dt parameter is a well-documented proxy for left ventricular contractility.

In current practice, external ultrasound measurements are used to calculate dP/dt. Such external ultrasound is used to observe wall motion directly. Most commonly, the ultrasound operator uses the ultrasound system in a tissue Doppler mode, a feature known as tissue Doppler imaging (TDI), to evaluate the time course of displacement of the septum relative to the left ventricle free wall. The current view of clinicians is that ultrasonographic evaluation using TDI or a similar approach may become an important part of qualifying patients for CRT therapy.

As currently delivered, CRT therapy is effective in about half to two-thirds of patients implanted with a resynchronization device. In approximately one-third of these patients, this therapy provides a two-class improvement in patient symptoms as measured by the New York Heart Association scale. In about one-third of these patients, a one-class improvement in cardiovascular symptoms is accomplished. In the remaining third of patients, there is no improvement or, in a small minority, a deterioration in cardiac performance. This group of patients is referred to as non-responders. It is possible that the one-class New York Heart Association responders are actually marginal or partial responders to the therapy, given the dramatic results seen in a minority.

The synchronization therapy, in order to be optimal, targets the cardiac wall segment point of maximal delay, and advances the timing to synchronize contraction with an earlier contracting region of the heart, typically the septum. However, the current placement technique for CRT devices is usually empiric. A physician will cannulate a vein that appears to be in the region described by the literature as most effective. The device is then positioned, stimulation is carried out, and the lack of extra-cardiac stimulation, such as diaphragmatic pacing, is confirmed. With the currently available techniques, rarely is there time or means for optimizing cardiac performance.

When attempted today, clinical CRT optimization must be preformed by laborious manual method of an ultrasonographer evaluating cardiac wall motion at different lead positions and different interventricular delay (IVD) settings. The IVD is the ability of pacemakers to be set up with different timing on the pacing pulse that goes to the right ventricle versus the left ventricle. In addition, all pacemakers have the ability to vary the atrio-ventricular delay, which is the delay between stimulation of the atria and the ventricle or ventricles themselves. These settings can be important in addition to the location of the left ventricular stimulating electrode itself in resynchronizing the patient.

Current use of Doppler to localize elements in the heart have been limited to wall position determination via external ultrasonography, typically for purposes of measuring valve function, cardiac output, or rarely, synchronization index.

There is currently no useful clinically available means of determining optimal CRT settings on a substantially automatic or a real-time, machine readable basis. It would be an important advancement in cardiology to have an implantable means of monitoring the mechanical performance of the heart in real time, an immediate application being in setting the functions of cardiac resynchronization therapy pacemakers, with further application to the pharmacologic management of heart failure patients, arrhythmia detection and ischemia detection, etc.

### Relevant Literature

Publications of interest include: U.S. Pat. Nos. 6,795,732; 6,625,493; 6,044,299; 6,002,963; 5,991,661; 5,772,108; 5,983,126 and 5,544,656; as well as United States Published Patent Application No. 2005/0038481.

### SUMMARY OF THE INVENTION

Methods for evaluating tissue location motion, such as of a cardiac location, e.g., heart wall, via continuous field tomography are provided. In the subject methods, a continuous field (e.g., an electrical field) sensing element is stably associated with a tissue location, and a property of, e.g., a change in, the continuous field sensed by the sensing element is employed to evaluate movement of the tissue location. Also provided are systems, devices and related compositions for practicing the subject methods. The subject methods and devices find use in a variety of different applications, such as cardiac related applications, e.g., cardiac resynchronization therapy, and other applications.

As reviewed in greater detail below, embodiments of the present invention can use several types of continuous fields to facilitate the tomography methods of the present invention. For example, a tomography system may apply an electrical field, a magnetic field, or a pressure field (e.g., using acoustic waves), as a continuous field. In general, a dynamic field operating at a given frequency can be a traveling wave or a standing wave. The field is typically a vector quantity, whereas the field magnitude is often a scalar. Without losing generality, the field magnitude can be expressed as:

F₀=A·sin(2π·f·t+φ)

where A is the field amplitude, f is the frequency at which the field oscillates, t is the time, and Φ is the phase shift.

When a tissue region is subject to such a field, and when a sensing element, such as an electrode, resides in the same region (e.g., by being stably associated therewith), the field can induce a signal upon the sensing element. The induced signal may be of the form:

S=B·sin(2π·f′·t+φ′)

where B is the amplitude of the induced signal, f′ is the induced signal's frequency, and φ′ is the induced signal's phase shift. In certain embodiments, of interest is the a transformation function “T”, which can be determined from S and F_{O} using the following relationship: S=T(x,y,z,t)° F_{O}. In these embodiments, tissue location movement may be evaluated by detecting a transformation of the continuous field. Because B, f′ and φ′ may depend upon the sensing element's location or movement in the field, one can perform tomography based on one or more of these values.

For example, if a continuous electrical field driven by an alternating-current (AC) voltage is present in a tissue region, one may detect an induced voltage on an electrode therein. The frequency of the induced voltage, f′ is the same as the frequency of the electrical field. The amplitude of the induced signal, however, varies with the location of the electrode. By detecting the induced voltage and by measuring the amplitude of the signal the location as well as the velocity of the electrode can be determine.

A magnetic field can achieve a similar result. For example, an AC sinusoidal current passing through a coil can produce a dynamic magnetic field which also changes at the same frequency. When an electrode containing an inductor coil is present in this magnetic field, a current is induced in the inductor coil. Consequently, by detecting the induced current, the location of the electrode can be determine.

A pressure field based on acoustic wave can also facilitate measurement of an sensing element's motion. An ultrasonic wave is directed to a tissue region. The ultrasonic wave can easily propagate through the tissue. A moving sensing element within the tissue may receive the ultrasonic wave with a Doppler frequency shift. As a result, by measuring the amount of Doppler frequency shift, the direction and velocity of the electrode's movement can be determined.

In general, continuous field tomography can be based upon measurement of the amplitude, frequency, and phase shift of the induced signal. When the external field is an electrical field or a magnetic field, the induced signal's amplitude is the main property for consideration in representative embodiments. When the external field is a pressure field, the induced signal's frequency is the main property for consideration in representative embodiments. The description below provides various embodiments of the present invention in detail.

### BRIEF DESCRIPTION OF THE FIGURES

FIGS. 1 to 8 provide depictions of various electrical tomography system embodiments of the subject invention.

FIGS. 9 and 10 provide depictions of various magnetic tomography system embodiments of the subject invention.

FIG. 11 provides a graphical result of the data obtained in the Pig Study experiment, described below.

FIG. 12 provides a diagram of a representative embodiment of the implantable Doppler tomography system.

FIG. 13 provides a diagram of an additional embodiment of the inventive implantable Doppler tomography system.

FIG. 14 provides a three dimensional cutaway view of placement of and embodiment of the Doppler tomography system in the left ventricle.

FIG. 15 illustrates an exemplary configuration for electrical tomography, in accordance with an embodiment of the present invention.

FIG. 16 illustrates an exemplary configuration for 3-D electrical tomography, in accordance with an embodiment of the present invention.

FIG. 17 illustrates an exemplary configuration for magnetic tomography using one inductor coil, in accordance with an embodiment of the present invention.

FIG. 18 illustrates an exemplary configuration for 3-D magnetic tomography using a magnetic gradiometer, in accordance with an embodiment of the present invention.

FIG. 19 illustrates an electrical tomography system based on an existing pacing system, in accordance with an embodiment of the present invention.

FIG. 20 illustrates a schematic circuit diagram for the voltage-driving and data-acquisition system 1904 in FIG. 19, in accordance with an embodiment of the present invention.

FIG. 21 illustrates a configuration for driving electrodes to mitigate effects caused by large electrode interface impedance in an electrical tomography system, in accordance with an embodiment of the present invention.

FIG. 22 illustrates a schematic circuit diagram showing an exemplary implementation of a frequency-division-multiplexing system for simultaneously transmitting multiple electrical tomography signals over a single wire, in accordance with an embodiment of the present invention

FIG. 23 illustrates the locations of electrodes used in an experiment demonstrating the analysis of electrical tomography signals, in accordance with an embodiment of the present invention.

FIG. 24 presents the time-series plots for measured voltages of six target electrodes in the experiment as shown in FIG. 9, in accordance with an embodiment of the present invention.

FIG. 25 presents the time-series plots constructed based on the eigenvectors obtained in the experiment as shown in FIG. 9, in accordance with an embodiment of the present invention.

FIGS. 26-29 provide a view of an electrode configuration that finds use in electrical gradient tomography applications of the present invention, as well explanatory graphs and electric field maps therefore.

FIG. 30 provides a view of a device according to a representative embodiments of the invention.

### DESCRIPTION OF SPECIFIC REPRESENTATIVE EMBODIMENTS

Methods for evaluating motion of a tissue location, such as of a cardiac location, e.g., a heart wall location, via continuous field tomography are provided. In the subject methods, a continuous field (e.g., an electrical field) sensing element is stably associated with the tissue location(s) of interest, and a property of the continuous field, e.g., a change in the continuous field, sensed by the sensing element is employed to evaluate movement of the tissue location. Also provided are systems, devices and related compositions for practicing the subject methods. The subject methods and devices find use in a variety of different applications, e.g., cardiac resynchronization therapy.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and tie like in connection with the recitation of claim elements, or use of a negative“limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing the subject invention, a general overview of aspects of continuous field tomography is provided first. Next, representative embodiments of different representative types of continuous fields and applications based thereon are reviewed in greater detail, both generally and in terms of specific representative devices and systems that may be employed in such embodiments. Following this section, representative applications in which the subject invention finds use are described, as well as other aspects of the invention, such as computer related embodiments and kits that find use in practicing the invention.

### Overview of Continuous Field Tomography

As summarized above, the subject invention provides continuous field tomography methods for evaluating movement of a tissue location of interest. In the subject tomography methods, data obtained by a sensing element stably associated with the tissue location of interest as it moves through an applied continuous field are employed. While the methods may be viewed as tomography methods, such a characterization does not mean that the methods are necessarily employed to obtain a map of a given tissue location, such as a 2-dimensional or 3-dimensional map, but instead just that changes in a sensing element as it moves through an applied continuous field are used to evaluate or characterize a tissue location in some way.

By “continuous field tomography method” is meant a method which employs detected changes in an applied continuous field to obtain a signal, which signal is then employed to determine tissue location movement. For the purposes of this application, the term “continuous field” means a field from which tomography measurement data is obtained from the field's continuous aspect. The continuous field is one or more cycles of a sine wave. There is no necessary requirement for discontinuity in the field to obtain data. As such, the applied field employed in the subject invention is continuous over a given period of time.

The “continuous field” used for tomography measurement may, at times, be provided with disruptions or naturally have some disruptions, and still fall within the present meaning of “continuous field”. As clarifying examples, pulsing the field to conserve power or mutiplexing between different fields remains within the meaning of “continuous field” for the purposes of the present invention. In contrast, a time-of-flight detection method falls outside of the meaning of “continuous field” for the purposes of the present invention. Accordingly, the continuous field applied in the subject methods is distinguished from “time of flight” applications, in which a duration limited signal or series of such signals is emitted from a first location and the time required to detect the emitted signal at a second location is employed to obtain desired data. At best, if a series of signals are generated in a time of flight application, the series of signals is discontinuous, and therefore not a continuous field, such as the field employed in the present invention.

As summarized above, the subject invention provides methods of evaluating movement of a tissue location. “Evaluating” is used herein to refer to any type of detecting, assessing or analyzing, and may be qualitative or quantitative. In representative embodiments, movement can be determined relative to another tissue location, such that the methods are employed to determine movement of two or more tissue locations relative to each other.

The tissue location(s) is generally a defined location or portion of a body, i.e., subject, where in many embodiments it is a defined location or portion (i.e., domain or region) of a body structure, such as an organ, where in representative embodiments the body structure is an internal body structure, such as an internal organ, e.g., heart, kidney, stomach, lung, etc. In representative embodiments, the tissue location is a cardiac location. As such and for ease of further description, the various aspects of the invention are now reviewed in terms of evaluating motion of a cardiac location. The cardiac location may be either endocardial or epicardial, as desired, and may be an atrial or ventricular location. Where the tissue location is a cardiac location, in representative embodiments, the cardiac location is a heart wall location, e.g., a chamber wall, such as a ventricular wall, a septal wall, etc. Although the invention is now further described in terms of cardiac motion evaluation embodiments, the invention is not so limited, the invention being readily adaptable to evaluation of movement of a wide variety of different tissue locations.

In practicing embodiments of the invention, following implantation of any required elements in a subject (e.g., using known surgical techniques), the first step is to set up or produce, i.e., generate, a continuous field in a manner such that the tissue location(s) of interest is present in the generated continuous field. In certain embodiments, a single continuous field is generated, while in other embodiments a plurality of different continuous fields are generated, e.g., two or more, such as three or more, where in certain of these embodiments, the generated continuous fields may be substantially orthogonal to one another.

In practicing the subject methods, the applied continuous field may be applied using any convenient format, e.g., from outside the body, from an internal body site, or a combination thereof, so long as the tissue location(s) of interest resides in the applied continuous field. As such, in certain embodiments the applied continuous field is applied from an external body location, e.g., from a body surface location. In yet other embodiments, the continuous field is generated from an internal site, e.g., from an implanted device.

In the subject methods, following generation of the applied continuous field, as described above, a signal (representing data) from a continuous field sensing element that is stably associated with the tissue location of interest is then detected to evaluate movement of the tissue location. In representative embodiments, a signal from the sensing element is detected at least twice over a duration of time, e.g., to determine whether a parameter(s) being sensed by the sensing element has changed or not over the period of time, and therefore whether or not the tissue location of interest has moved over the period of time of interest. In certain embodiments, a change in a parameter is detected by the sensing element to evaluate movement of the tissue location. In certain embodiments, the detected change may also be referred to as a detected “transformation,” as defined above.

In representative embodiments, at least one parameter of the applied continuous field is detected by the sensing element at two or more different times. Parameters of interest include, but are not limited to: amplitude, phase and frequency of the applied continuous field, as reviewed in greater detail below. In certain embodiments, the parameter of interest is detected at the two or more different times in a manner such that one or more of the other of the three parameters is substantially constant, if not constant.

By “stably associated with” is meant that the sensing element is substantially if not completely fixed relative to the tissue location of interest such that when the tissue location of interest moves, the sensing element also moves. As the employed continuous field sensing element is stably associated with the tissue location, its movement is at least a proxy for, and in certain embodiments is the same as, the movement of the tissue location to which it is stably associated, such that movement of the sensing element can be used to evaluate movement of the tissue location of interest. The continuous field sensing element may be stably associated with the tissue location using any convenient approach, such as by attaching the sensing element to the tissue location by using an attachment element, such as a hook, etc., by having the sensing element on a structure that compresses the sensing element against the tissue location such that the two are stably associated, etc.

In a given embodiment, the sensing element can provide output in an interval fashion or continuous fashion for a given duration of time, as desired.

In certain embodiments, a single sensing element is employed. In such methods, evaluation may include monitoring movement of the tissue location over a given period of time. In certain embodiments, two or more distinct sensing elements are employed to evaluate movement of two or more distinct tissue locations. The number of different sensing elements that are employed in a given embodiment may vary greatly, where in certain embodiments the number employed is 2 or more, such as 3 or more, 4 or more, 5 or more, 8 or more, 10 or more, etc. In such multi-sensor embodiments, the methods may include evaluating movement of the two or more distinct locations relative to each other.

In certain embodiments, the subject methods include providing a system that includes: (a) a continuous field generation element; and (b) a continuous field sensing element that is stably associated with the tissue location of interest. This providing step may include either implanting one or more new elements into a body, or simply employing an already existing implanted system, e.g., a pacing system, e.g., by using an adapter (for example a module that, when operationally connected to a pre-existing implant, enables the implant to perform the subject methods), as, described below. This step, if employed, may be carried out using any convenient protocol, where a variety of protocols are well known to those of skill in the art.

The subject methods may be used in a variety of different kinds of animals, where the animals are typically “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g., rabbits) and primates (e.g., humans, chimpanzees, and monkeys). In many embodiments, the subjects or patients will be humans.

The tissue movement evaluation data obtained using the subject methods may be employed in a variety of different applications, including but not limited to monitoring applications, treatment applications, etc. Representative applications in which the data obtained from the subject methods finds use are further reviewed in greater detail below.

With respect to the subject methods, the nature of the applied continuous field employed in the subject methods may vary depending on the particular application. The inventive continuous field tomography devices and methods enjoy a rich diversity of technical approaches. By example, an extraordinarily broad range of continuous field sources can be utilized in the inventive devices to make tomography measurement of the structure and movement of internal anatomical features. Electric, magnetic, acoustic, pressure waves, light and even heat can be utilized to provide this uniquely informative clinical information.

In representative embodiments, the continuous field that is applied is a wave field. In representative embodiments, the wave field is an electromagnetic wave. Representative electromagnetic continuous fields of interest electrical and magnetic fields, as well as light. In yet other representative embodiments, the wave is a pressure wave, where a representative continuous field of this type is an acoustic field.

From changes determined in these measurements obtained from the continuous field sensing element, the dynamics and timing of tissue movement can be derived. This rich source of data allows the generation of both physical anatomical dimensions and the physiological functions which they bespeak, typically in real time.

Each of the methods within the broad diversity of continuous field tomography approaches has unique characteristics which optimize the strengths of a particular continuous field source and special features that make them optimally useful in a particular application. The wealth of data produced by this range of devices provides clinicians and other health care providers, as well as the patients themselves, with unprecedented medical information of high value in the medical armamentarium.

While the specific device approaches within the broad family of continuous field tomography devices has considerable range and diversity, they share core commonalities. These core commonalities are often most apparent in how the signals are processed by circuitry, software and firmware providing the raw data collection, processing and graphic display of data.

The underlying precept among continuous field tomography methods is that a source is provided which generates a field ψ. ψ varies throughout the internal anatomical area of interest.

One example of the source field ψ can be expressed in a form:

ψ=A sin(2πft+φ)

where:

f is the frequency,

φ is a phase,

A is the amplitude, and

t is time.

In certain embodiments, the field oscillates as a function of time, and can be described simply an AC field.

The field can be used in a number of different embodiments to provide anatomical tomography data. By example, the field can be selected from an electric field, a magnetic field, a pressure field (e.g., a sound field), or a light field, or a thermal field, among others. It could also be a combination of various fields, such as in the case of an electro-magnetic field.

A core feature of gleaning data from the broad range of useful continuous fields is that either A, f or φ is a function of some parameter(s) of interest. Two representative parameters of interest among the many available parameters are location position and location velocity. When one or more properties of the field, e.g., A, f and/or φ, is sampled at various points, and the measured property is compared to the reference value, interesting information can be extracted from these raw data, and important information obtained.

The various approaches to detecting the change in the property of interest demonstrates the flexibility and breadth of the inventive concept. Change in amplitude or phase can be determined using standard approaches, such as through lock-in detection. In the case of the lock-in approach, a single phase lock is used to detect the amplitude change. If the device is provided with a dual phase lock-in, the phase change can be detected. There are also other ways of detecting phase change which are specific to the type of field, discussed elsewhere in the present application. Where respect to frequency, any convenient method for detecting frequency shift, such as small frequency shift, may be employed, such as FM demodulation. FM demodultion is the frequency demodulation similar to what is provided in an FM radio. In this way the source field is the carrier frequency and the small shifts in frequency can be identified in the demodulated signal.

Table 1 shows some of the range of fields and variable field characteristics or properties which can be employed in the present invention. The generalized inventive concept demonstrated in Table 1 provides a framework for the ordinary skilled artisan to produce a wide range of embodiments of the present inventive device, selecting those features within this framework which are most advantageous to a particular clinical need or physical environment. Table 1 provides a generalized 3×5 matrix for considering different features when selecting among the range of inventive embodiments those best suited to a particular need.

TABLE 1

| | 1 | 2 | 3 | 4 | 5 |
|---|---|---|---|---|---|
| | Electrical | Magnetic | Accoustic | Light | Heat |
| Amplitude | voltage | magnetic | pressure | wave | Δ gradient |
| | field | field | | | |
| Phase | voltage | magnetic | pressure | wave | Δ gradient |
| | field | field | | | |

| | | | | |
|---|---|---|---|---|
| Frequency | Electro-Magnetic | Doppler | Relativistic | n/a |
| | Doppler | | Doppler | |

### Continuous Field Tomography Matrix

### field source and wave sampling

In Table 1, the top row provides the various representative types of continuous fields that can be selected, such as electric, magnetic, sound, light and thermal (i.e., heat) fields, where this list is not exhaustive. The various rows are the field properties which can be detected by the continuous field sensing element. Many properties, such as amplitude, phase or frequency, among others, and various combinations of different properties can be selected.

As examples of the general applicability of the inventive insight, the following section provides representative embodiments of how amplitude and phase in electric and magnetic tomography can be determined by using a lock-in amplifier. By considering the teachings of the present invention, the ordinary skill artisan, without undue experimentation, will be able to best select the embodiment of the continuous field tomography invention best suited to the clinical data need to be addressed.

It is noted that the while the subject invention is directed to continuous field tomographic methods of evaluating a movement of a tissue location of interest and is reviewed herein by multiple different and distinct embodiments that fully support the broad continuous field tomographic approach, each representative continuous field tomographic embodiment reviewed below is of interest in its own right, depending on a particularly application. Furthermore, while certain embodiments reviewed below are described in terms of use in CRT applications, such should not be viewed as limiting, as such description is merely done in order to easily describe the aspect of the invention of interest, the inventive approach to tissue movement evaluation having broad applicability beyond CRT.

### Electrical Tomography

As summarized in Table 1, electrical tomography embodiments of the subject invention employ a voltage field as the applied continuous field. Following an overview of electrical tomography provided below, a number of specific representative embodiments are reviewed in greater detail.

### Overview of Electrical Tomography

In practicing electrical tomographic embodiments of the invention, following implantation of any required elements in a subject (e.g., using known surgical techniques) the first step is to set up or produce, i.e., generate, an electric field in a manner such that the tissue location of interest is present in the generated electric field. In certain embodiments, a single electric field is generated, while in other embodiments a plurality of different electric fields are generated, e.g., two or more, such as three or more, where in these embodiments, the generated electric fields may or may not be substantially orthogonal to one another. The electric field or fields employed in the subject methods may be produced using any convenient electric field generation element, where in certain embodiments the electric field is set up between a driving electrode and a ground element, e.g., a second electrode, an implanted medical device that can serve as a ground, such as a “can” of an implantable cardiac device (e.g., pacemaker), etc. The electric field generation elements may be implantable such that they generate the electric field from within the body, or the elements may be ones that generate the electric field from locations outside of the body, or a combination thereof.

In certain embodiments, the continuous electric field is a radiofrequency or RF field. As such, in these embodiments, the electric field generation element generates an alternating current electric field, e.g., that comprises an RF field, where the RF field has a frequency ranging from about 1 kHz to about 100 GHz or more, such as from about 10 kHz to about 10 MHz, including from about 25 KHz to about 1 MHz. Aspects of this embodiment of the present invention involve the application of alternating current within the body transmitted between two electrodes with an additional electrode pair being used to record changes in a property, e.g., amplitude, within the applied RF field. Several different frequencies can be used to establish different axes and improve resolution, e.g., by employing either RF energy transmitted from a subcutaneous or cutaneous location, in various plains, or by electrodes, deployed for example on an inter-cardiac lead, which may be simultaneously used for pacing and sensing. Where different frequencies are employed simultaneously, the magnitude of the difference in frequencies will, in certain embodiments, range from about 100 Hz to about 100 KHz, such as from about 5 KHz to about 50 KHz. Amplitude information can be used to derive the position of various sensors relative to the emitters of the alternating current.

In the subject methods, following generation of the electric field, as described above, a signal from an electric field sensing element that is stably associated with the tissue location of interest is then detected, e.g., at least twice over a duration of time, to evaluate movement of said tissue location. As the employed electric field sensing element is stably associated with the tissue location, its movement is the same as the movement of the tissue location to which it is stably associated.

The electric field sensing element may be stably associated with the tissue location using any convenient approach, such as by attaching the sensing element to the tissue location by using an attachment element, such as a hook, etc., by having the sensing element on a structure that compresses the sensing element against the tissue location such that the two are stably associated, etc. In certain embodiments, two or more different sensing elements are employed at different tissue locations. The number of different sensing elements that are employed in a given embodiment may vary greatly, where in certain embodiments the number employed is 2 or more, such as 3 or more, 4 or more, 5 or more, 8 or more, 10 or more, etc.

The sensing element is, in representative embodiments, an electric potential sensing element, such as an electrode. In these embodiments, the sensing element provides a value for a sensed electric potential which is a function of the location of the sensing element in the generated electric field. As the tissue location with which the sensing element is stably associated moves, the electric potential sensed by the sensing element varies. The electric potential that is sensed by the sensing element is provided as a voltage in many representative embodiments. As such, a change in voltage output sensed by the sensing element between two different times provides for evaluation of movement of the tissue location over a duration of time that includes the two different times.

In certain embodiments, one detects the change of the magnitude of the received signal. One simple embodiment is to employ a peak detector circuit that would essentially follow the maximum voltage, essentially tracking the top of this curve. An alternative would be an envelope detector that would basically measure the difference between the top of the curve and the bottom of the curve. As both of these techniques are susceptible to noise, a lock-in amplifier can be employed as desired to discriminate between the received signal and the noise. The lock-in amplifier is a specific embodiment of a technique called synchronous detection. Other kinds of synchronous detection would be applicable to this method. Another form of synchronous detection is amplitude modulated radio detection. An AM radio receiver consists of an electronic circuit that is designed to extract the amplitude of an envelope from the received wave form that may contain noise.

In representative embodiments, the amplitude approach is used to determine the relative motion of different walls of the heart with respect to one another. For example, where the electric field is an RF field, either an externally applied or subcutaneously applied RF field or different electrode pairs may be used as emitters at different frequencies with other electrodes simultaneously recording voltages. In such a way, multiple lines of position may be obtained, one relative to another, and a timing plot described, demonstrating movement of different wall segments with respect to each other. This information can be correlated with markers of the cardiac cycle such as the R wave, or other electrical activity, or the pressure signal, or other mechanical measures, in order to obtain a timing plot demonstrating the synchrony of the heart. Of interest is the fact that in this application the intent is to determine the relative position of the catheters and corresponding wall segments of the heart with respect to each other in the time domain, e.g., in order to determine synchrony. In this manner the present invention is much more resistant to the effects of noise or changes in the local impedance environment than other methods.

In certain embodiments, the methods and systems only determine the relative timing and distance along the line of position of, for example, two electrodes, one with respect to another. By using multiple frequencies and multiple electrodes pairs, multiple lines of position can be derived, improving the resolution of this system with respect to determining the inter-ventricular and/or intra-ventricular synchrony of a given heart.

In a given embodiment, the sensing element can provide output in an interval fashion or continuous fashion for a given duration of time, as desired.

In certain embodiments, a single sensing element is employed. In such methods, evaluation may include monitoring movement of the tissue location over a given period of time.

In certain embodiments, two or more distinct sensing elements are employed to evaluate movement of two or more distinct tissue locations. In such embodiments, the methods may include evaluating movement of the two or more distinct locations relative to each other.

A feature of representative embodiments of the invention is that the evaluation step employed does not include an impedance determination step, and the signal employed is not an impedance signal. As such, the methods are not impedance based methods in which the impedance of current between points is determined and then employed to make a given evaluation. As such, the methods of these embodiments are not impedance based methods as described in published United States patent application 2005/0038481.

As depicted in Table 1 above, a number of different properties of the continuous field may be detected to provide data for the evaluation of tissue location movement, where representative properties of interest are amplitude, phase and frequency.

### Amplitude

In electrical tomography applications, the field ψ is a voltage generated by two electrodes. In representative embodiments, an AC voltage is applied between the two electrodes. The amplitude (e.g., as detected by a sensing electrode) of this voltage field then varies as a function of position.

How the amplitude of the voltage field varies depends on the particulars of the medium. In free space for example, the voltage field varies as 1/R in the near field of each electrode and 1/R³ in the far field, r being the distance from each electrode. However, in practical application, the intervening body tissues, fluids and spaces of different electrical permittivity influence the raw form of A.

In representative electrical tomographic embodiments of the present invention, two electrodes are employed to generate the electrical field. A third electrode is then provided to sense the various positions of interest. In representative embodiments, a lock-in detector is locked into the same frequency f upon which the field was generated. This allows determination of the amplitude, as represented in the following formula:

V(t,\vec{r})=A(\vec{r})sin(2πft+φ); f, φ fixed allow lock-in detection

In this manner, the electrical tomography embodiment of the present invention achieves a very high precision in the face of external noise sources.

The electrodes are in conductive contact with the tissues of the body. As a result, the electrodes force a voltage on their surface. Because the tissues are conductive, the voltage induces an electric field in the tissues. This causes current to flow through the tissues of the body.

Through the impedances of the tissues of the body, this flow in current generates a voltage gradient; essentially an AC voltage gradient. When this occurs, high impedance sensing electrodes can, measure this voltage gradient. The voltage gradient is then demodulated.

### Phase

Moving to other approaches as shown in Table 1, electrical tomography is accomplished equally as well using phase detection. In this case, as a sense electrode moves in the field, e.g., as generated by the drive electrodes, the phase of the field detected by the sense electrode changes. By example of how this particular embodiment operates, note that for low frequencies on the order of 100 Kilohertz, the phase change is very small. However, the phase change becomes larger at higher frequencies. Therefore, in clinical applications where higher frequencies are of interest, detecting phase change, rather than amplitude change, can be a method of interest.

The above examples of electrical tomography methods of the present invention provide an overview of some aspects of these embodiments of the present invention. This overview is provided to show an example of the core commonality of the many embodiments contemplated by the subject invention There are multiple embodiments conceived by the present inventors for electrical tomography methods. The above summarized embodiments are provided for illustrative purposes only in order to demonstrate how electrical tomography ties into the over arching theme of the present invention.

### Electrical Tomography Representative Methods/Systems/Devices

In one aspect of the invention, a system is employed that includes an electric field generation element and a sensing element for sensing changes in the electric field, where the sensing element is stably associated with a cardiac location of interest, e.g., a heart wall, such as a ventricular wall, septal wall, etc., such that changes in detected electrical field by the sensing element can be correlated with movement of the cardiac location of interest. The system is used to generate an electrical field between a reference and a driver electrode (signal generator or generator of applied electric field). A third sensing electrode, e.g., intracardiac sensing electrode (signal receiver), is used to measure the amplitude of the electric field. Any change in position of this intracardiac sensing electrode relative to the reference and driver electrodes causes a related change in sensed voltage amplitude. Thereby the motion of the electrodes relative to one another can be determined (e.g., by a signal processor) and provide cardiac mechanical contractile magnitude and timing information (e.g., output to a signal display) such as initiation of a systolic contraction. In representative embodiments, the system is comprised of the following main components: 1) three or more electrodes with at least one electrode being intracardiac (e.g., the sensing electrode); 2) a signal generator; 3) a signal receiver (where the signal generator and receiver work together to produce the applied electric field; 4) a signal processor; and 5) a signal display. For CRT applications, in order to optimize CRT in real-time, the electrodes can alternate back and forth between pacing and motion sensing functions.

This approach can be extended to pacing leads with a plurality of sensing electrodes placed around the heart, which provides a more comprehensive picture of the global and regional mechanical motion of the heart. With multiple electrodes, artifacts such as breathing can be filtered out. Furthermore, multiple electrodes provide three-dimensional relative or absolute motion information by having electrodes switching between the roles of reference, driver, or sense electrode. Indeed any of the electrodes (including a pacemaker can) in this system can be used as a reference, driver, or sense electrode.

This approach can be further extended to employ a variety of electrical field generating elements, creating distinct electrical fields in each of multiple planes. Sensing electrodes simultaneously report amplitude from each of the multiplanar electrical fields, thereby improving resolution in characterizing intracardiac wall motion. Using such resolution-enhancing embodiments can, with proper calibration, yield parameters, including stroke volume and ejection fraction, which are important in CHF management.

Another extension of this approach is to generate more than one electrical field in each plane through the use of the several driving electrodes. In this application, each co-planar electrical field is tailored to exploit different propagation characteristics within the human body. In this way, in addition to wall motion, valuable information can be obtained about the composition of the local fluids and tissues. Such data is clinically important in determining, without limitation, pulmonary congestion, myocardial thickness and hemodynamic parameters such as ejection fraction.

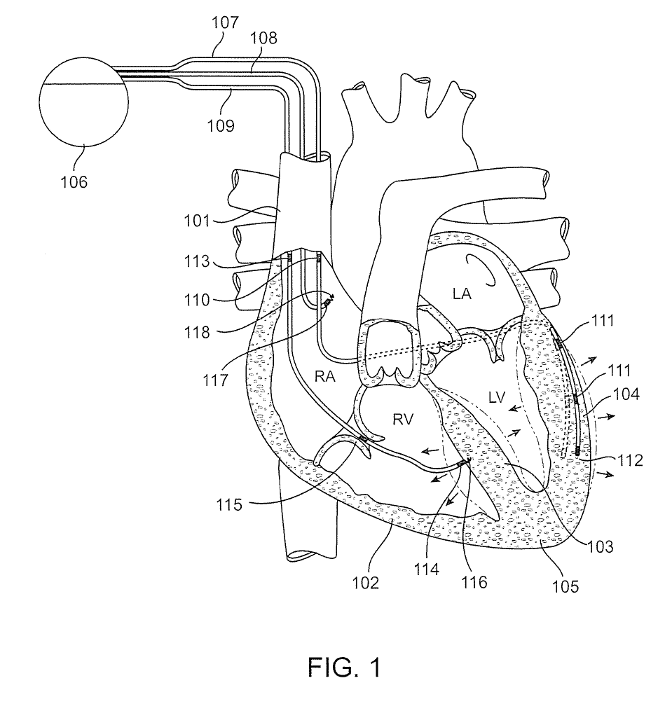

FIG. 1 provides a cross-sectional view of the heart with of an embodiment of the inventive electrical tomographic device, e.g., as embodied in a cardiac timing device, which includes a pacemaker 106, a right ventricle electrode lead 109, a right atrium electrode lead 108, and a left ventricle cardiac vein lead 107. Also shown are the right ventricle lateral wall 102, interventricular septal wall 103, apex of the heart 105, and a cardiac vein on the left ventricle lateral wall 104.

The left ventricle electrode lead 107 is comprised of a lead body and one or more electrodes 110,111, and 112. The distal electrodes 111 and 112 are located in the left ventricle cardiac vein and provide regional contractile information about this region of the heart. Having multiple distal electrodes allows a choice of optimal electrode location for CRT. The most proximal electrode 110 is located in the superior vena cava in the base of the heart. This basal heart location is essentially unmoving and therefore can be used as one of the fixed reference points for the cardiac wall motion sensing system.

In a representative embodiment, electrode lead 107 is constructed with the standard materials for a cardiac lead such as silicone or polyurethane for the lead body, and MP35N for the coiled or stranded conductors connected to Pt—Ir (90% platinum, 10% iriudium) electrodes 110, 111 and 112. Alternatively, these device components can be connected by a multiplex system (e.g., as described in published United States Patent Application publication nos.: 20040254483 titled “Methods and systems for measuring cardiac parameters”; 20040220637 titled “Method and apparatus for enhancing cardiac pacing”; 20040215049 titled “Method and system for remote hemodynamic monitoring”; and 20040193021 titled “Method and system for monitoring and treating hemodynamic parameters; the disclosures of which are herein incorporated by reference), to the proximal end of electrode lead 107. The proximal end of electrode lead 107 connects to a pacemaker 106.

The electrode lead 107 is placed in the heart using standard cardiac lead placement devices which include introducers, guide catheters, guidewires, and/or stylets. Briefly, an introducer is placed into the clavicle vein. A guide catheter is placed through the introducer and used to locate the coronary sinus in the right atrium. A guidewire is then used to locate a left ventricle cardiac vein. The electrode lead 107 is slid over the guidewire into the left ventricle cardiac vein 104 and tested until an optimal location for CRT is found. Once implanted a multi-electrode lead 107 still allows for continuous readjustments of the optimal electrode location.

The electrode lead 109 is placed in the right ventricle of the heart with an active fixation helix at the end 116 which is embedded into the cardiac septum. In this view, the electrode lead 109 is provided with one or multiple electrodes 113,114,115. The distal tip of the electrode lead 109 is provided with an active fixation helix 116 which is screwed into the mid-septum 103.

Electrode lead 109 is placed in the heart in a procedure similar to the typical placement procedures for cardiac right ventricle leads. Electrode lead 109 is placed in the heart using the standard cardiac lead devices which include introducers, guide catheters, guidewires, and/or stylets. Electrode lead 109 is inserted into the clavicle vein, thru the superior vena cava, through the right atrium and down into the right ventricle. Electrode lead 109 is positioned under fluoroscopy into the location the clinician has determined is clinically optimal and logistically practical for fixating the electrode lead 109 and obtaining motion timing information for the cardiac feature area surrounding the attachment site. Under fluoroscopy, the active fixation helix 116 is advanced and screwed into the cardiac tissue to secure electrode lead 109 onto the septum.

Once the electrode lead 109 is fixed on the septum, electrode lead 109 provides timing data for the regional motion and/or deformation of the septum. The electrode 115 which is located more proximally along electrode lead 109 provides timing data on the regional motions in those areas of the heart. By example, an electrode 115 situated near the AV valve, which spans the right atrium in the right ventricle, provides timing data regarding the closing and opening of the valve. The proximal electrode 113 is located in the superior vena cava in the base of the heart. This basal heart location is essentially unmoving and therefore can be used as one of the fixed reference points for the cardiac wall motion sensing system.

The electrode lead 109 is typically fabricated of a soft flexible lead with the capacity to conform to the shape of the heart chamber. The only fixation point in this embodiment of the present cardiac timing device is the active fixation helix 116 which is attaching the electrode lead 109 to the cardiac septum.

The electrode lead 108 is placed in the right atrium using an active fixation helix 118. The distal tip electrode 118 is used to both provide pacing and motion sensing of the right atrium.

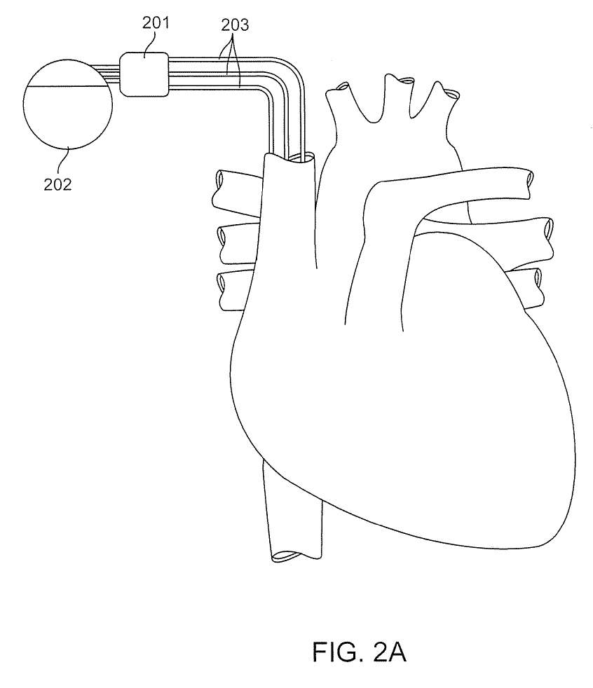

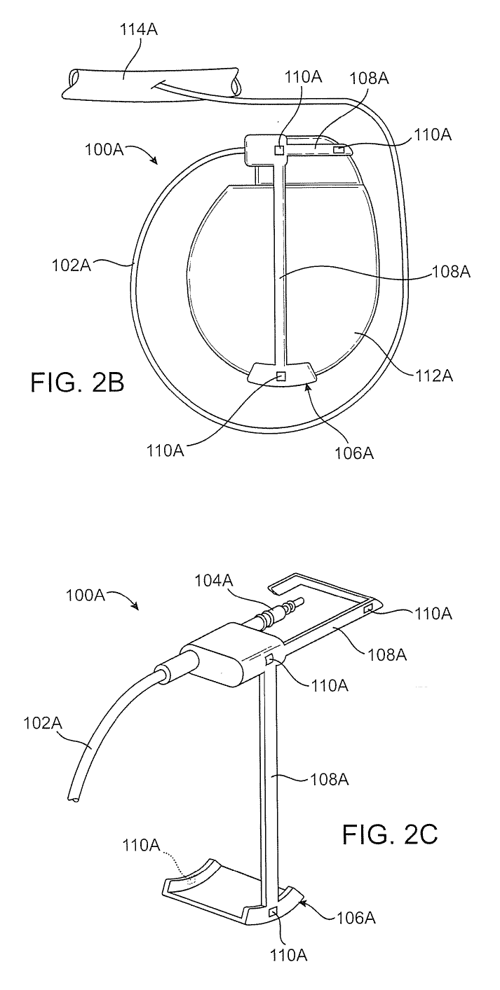

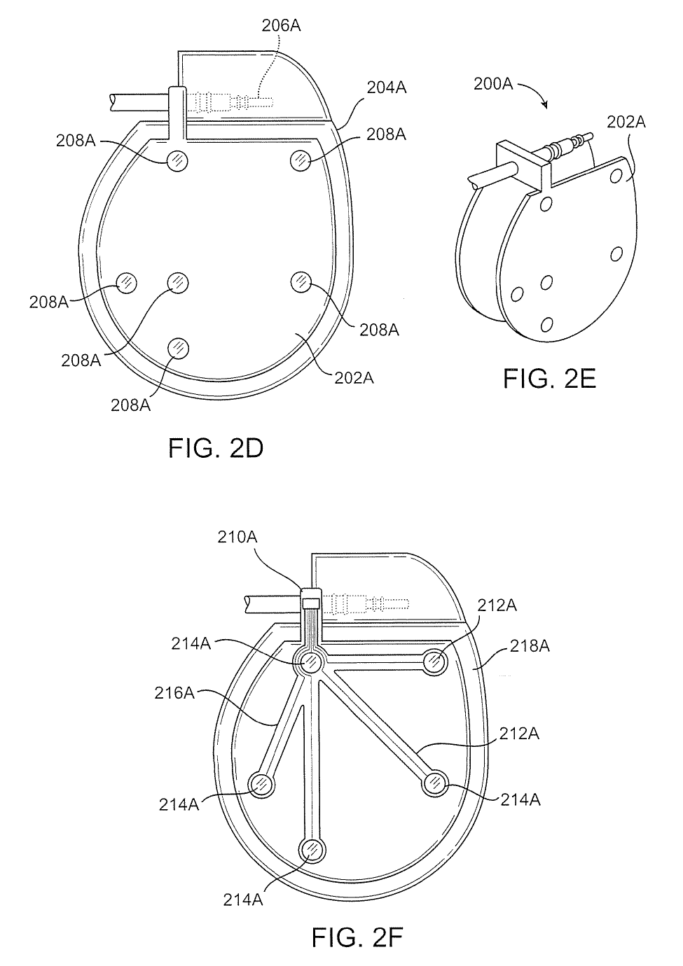

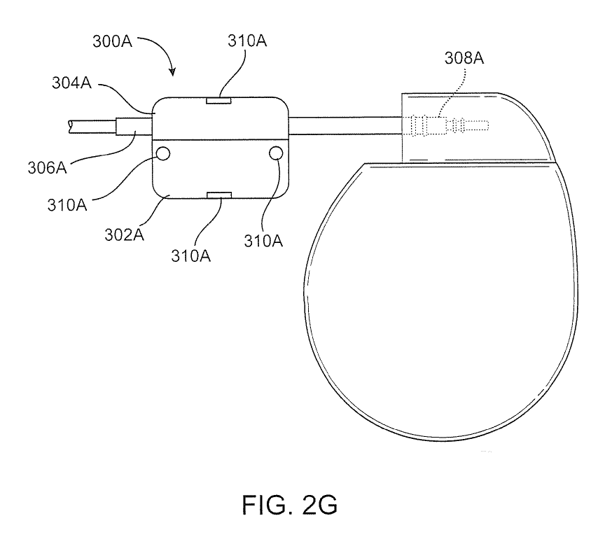

FIG. 2A provides a view of an additional of the embodiment described in FIG. 1 with an add-on module 201 which is connected in series in between pacemaker 202 and the electrode leads 203. The add-on module (i.e., adaptor) is comprised of a hermetically sealed housing which contains all the software, hardware, memory, wireless communication means, and battery necessary to run the cardiac wall motion sensing system. The housing is made of titanium and can be used as the reference electrode. On the proximal end, the add-on module 201 has lead type proximal connectors which can plug into the pacemaker header. On the distal, the add-on module 201 provides connectors for electrode leads 203. One of the main advantages of this embodiment is that it can be used with any commercial pacemaker. Even patients who already have a pacemaker and lead system implanted can benefit from this add-on module 201. In an outpatient setting and using a local anesthetic a small incision is made expose the subcutaneously implanted pacemaker. The leads 203 are then disconnected from the pacemaker and connected to the add-on module 201 which in turn is plugged into the pacemaker header. The incision is then sutured close and the patient can now immediately benefit from the cardiac motion sensing system.

Another embodiment of an add-on module is depicted in FIGS. 2B-2G, which module provides for one or more additional electrode sites, where the add-on module can be configured, as desired, to be employed with other implantable devices, such as pacemakers, to provide for the electrode field(s) desired for a given application. The electrode add-on module can include one or more electrodes, e.g., 2 or more, 3 or more, 4 or more, 5 or more, etc., as well as electrode pairs, e.g., 2 or more pairs, 3 or more pairs, 4 or more pairs, 5 or more pairs, as desired. Typically, the add-on module is configured or designed to be implantable, e.g., in a convenient subcutaneous location, and in certain embodiments may be configured to associate with, e.g., attached to, snap on to, etc., another implantable device, such as a pacemaker. As such, embodiments of the add-on modules provide additional electrode sites within the subcutaneous area near the pacemaker, and can be very easily and quickly placed during the implantation procedure.

In one representative as shown in FIGS. 2B and 2C, the device 100A is comprised of an electrode lead 102A inserted into a subclavian vein 114A with on the proximal end an IS-1, IS-4 or other connector 104A and a multielectrode clip-type device 106A with flexible struts 108A. The electrodes 110A can be positioned on all sides of the pacemaker can 112A to generate electrical fields in any direction for the ET method described previously. One advantage is that the position of all the electrodes 110A relative to each is fixed and known. Furthermore, the anatomical location of the device 100A is quite repeatable from one patient to the next which mitigates variability of the ET system between patients. In addition., the electrodes 110A, being located in a subcutaneous pocket, are removed from the problematic flow velocity induced changes in blood conductivity that affect electrical fields generated by the intravenous, atrium and ventricle electrodes. Also, the device 100A can be easily and quickly clipped directly onto the pacemaker to stabilize it.

The device 100A is also well adapted to work directly with a Protoplex™ lead and use the same Protoplex™ technology to select and activate various device electrodes 110A.

In another representative embodiment shown in FIGS. 2D and 2E, the device 200A is comprised of a low profile device 202A which slides into place around the front and/or back of the pacemaker 204A with minimal addition to the pacemaker volume. The IS-1, IS-4 or other connector 206A provides stability. The front and back portions include one or more electrodes 208A are used to generate electrical fields.

In another representative embodiment shown in FIG. 2F, the device 210A is also comprised of a very low profile “flex circuit” type device 212A with multiple electrodes 214A and conductors 216A, where the device is placed on and is connected to the pacemaker can 218A.

In another representative embodiment shown in FIG. 2G, the device 300A is comprised of a housing 302A containing electronics, RF telemetry, and battery, and a header 304A for the connectors of the electrode lead 306A and pacemaker can 308A. On the outside of the housing are located multiple electrodes 310A to generate multiple electrical fields. In certain-embodiments, this device could be used with standard leads, Protoplex™ leads, standard pacemakers, and/or ET enabled pacemakers.

The add-on modules of these embodiments can, in addition to providing one or more additional electrodes, be a platform device for various sensors such as temperature sensors, pressure sensors, and biosensors, as desired.

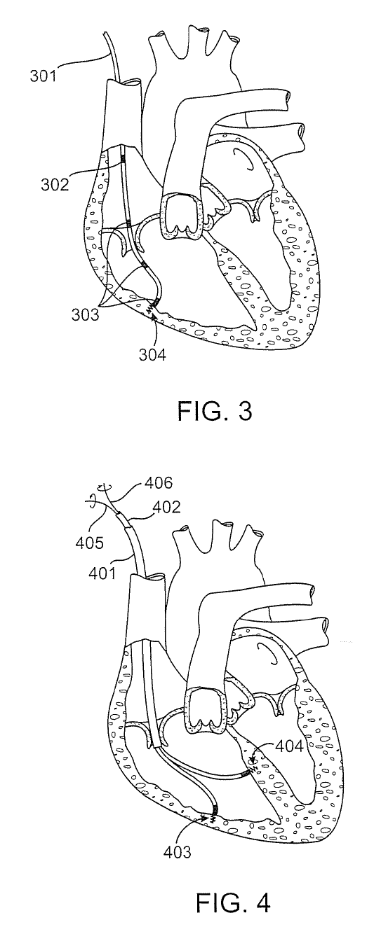

FIG. 3 provides a view of an electrode lead 301 with an active fixation helix on its distal end, but with a different site of attachment on the right ventricle lateral wall 304. Electrode lead 301 has one or more electrodes 303 along its length. Electrode lead 301 is physically identical to electrode lead 109 shown in FIG. 1. The primary difference between these two views is that in this view the distal end of the electrode lead is screwed into the lateral wall of the right ventricle 304 in order to obtain mechanical contractile magnitude and timing information of this region 304.

The clinical motivation for these fixation alternatives is to provide cardiac timing information via electrode leads 301 and 109 about the regional motions of the cardiac tissue where they are fixated. In FIG. 1, the electrode lead 109 attached to the septum provides cardiac timing data primarily for septal motion. In FIG. 3 electrode lead 301 is attached to the lateral wall of the right ventricle, and gives cardiac timing data primarily regarding the motion of that portion of the heart.

FIG. 4 provides a view of a bifurcated electrode lead 402 being placed with a guide catheter 401. In order to place the bifurcated electrode lead 402, the guide catheter 401 tip is first placed into the right ventricle and then the bifurcated electrode lead 402 is slowly advanced through the guide catheter 401. As the bifurcated electrode lead 402 enters into the right ventricle, it is released from the laterally confining guide catheter 401, and unfurls into its intrinsic bifurcated shape. Under fluoroscopy, bifurcated electrode lead 402 is advanced until the two distal tips 403 and 404 are in the desired location on the heart such as right lateral wall location 403 and septal wall location 404. Once distal tips 403 and 404 are in a desired position, torque wires 405 and 406 are used to advance the active fixation helixes and screw them into the tissue. Alternatively, passive fixation with tines can be employed to stabilize bifurcated electrode lead 402.

The inventive embodiment described in FIG. 4 enjoys number of advantages over the non-bifurcated embodiments. The bifurcated configuration of the inventive cardiac timing device allows, in a single deployment procedure, the placement of two active fixation helixes on two different regions of the heart. Thus, a considerable increase in cardiac timing information can be obtained in a single procedure. An additional advantage of this device configuration is that there is a more controlled reference position between distal tips 403 and 404 than are available with individual placement.

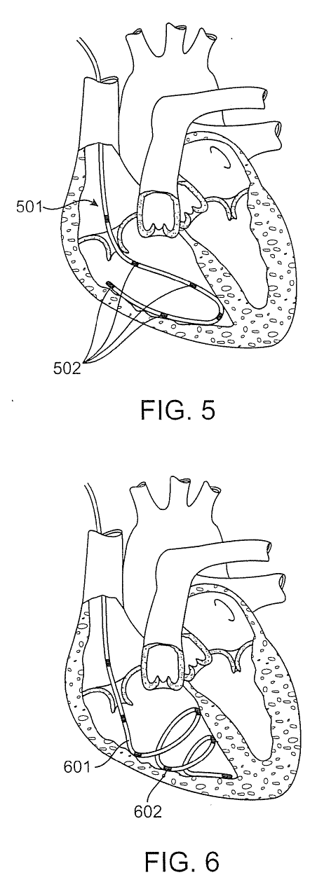

FIG. 5 provides a view of a U-shaped electrode lead 501. This diagram shows the position of U-shaped electrode lead 501 after deployment in the right ventricle. U-shaped electrode lead 501 is provided with one or more electrodes 502 along its length. The main motivation for U-shape configuration is to guarantee contact of the electrode lead with two walls of the heart, such as the septal wall and the right ventricle lateral wall.

U-shape electrode lead 501 is deployed using a guide catheter which is placed into the right ventricle. The straightened U-shape electrode lead 501 is then slowly advanced out of the guide catheter. As it exits the guide catheter, U-shape electrode lead 501 assumes its intrinsic U-shape within the right ventricle. Alternatively, a straight stylet placed within U-shape electrode lead 501 can be used to hold the lead in a straight position during initial right ventricle placement. Once the lead is placed in the right ventricle, the stylet is removed and U-shape electrode lead 501 assumes its intrinsic U-shape.

The fabrication of the U-shape can be accomplished through a number of known methods. By example, the silicone lead body can be molded as a U-shape during the processing. Alternatively, the metal conductor coils or strands within the lead body can be shape set into a U-shape using various heat treatment methods.

U-shaped electrode lead 501 may optionally include an active fixation helix (not shown) along the length of the lead to fixate it as shown in the figures above. However, such additional fixation need only be provided when there is an unusually demanding cardiac feature target area preferred for fixation, or the point of attachment needs to be highly precise. The most preferred embodiment of U-shaped electrode lead 501 does not require an active fixation, but by the nature of its U-shape will hold this position within the ventricle chamber of the heart.

In certain embodiments, during systole and diastole of the heart, the U-shaped electrode lead 501 flexes back and fourth and shifts slowly up and down. One of the advantages of the U shape is that it would give a direct measurement of contraction timing and magnitude of right ventricle by tracking the motions of the septal and right lateral ventricle wall.

FIG. 6 provides a view of spiral electrode lead 601. As with the examples above, spiral electrode lead 601 includes one or more electrodes 602 embedded along its length. Spiral lead 601 would be deployed using similar guide catheter and stylet methods as described for the U-shape electrode lead 501. As with U-shaped electrode lead 501, the primary purpose of the spiral shaped lead is to guarantee contact with the side walls of right ventricle chamber. In this case when the chamber contracts, the spiral lead would flex and a change in position would be measured between its one or more electrodes. The electrodes 602 provide regional timing and motion information at the various positions where the electrodes come in contact with the right ventricle walls. Another option is to have an active fixation helix on the distal tip, but in the preferred embodiment shown in FIG. 6, there is no active fixation.

As indicated, the above representative electrical tomography systems can be employed in a variety of different applications. A representative application in which the subject systems and methods find use is in the detection/monitoring of intraventricular and interventricular mechanical dyssynchrony, which characteristics are useful synchrony indices used for optimizing CRT (also known in the art as biventricular pacing). Intraventricular dyssynchrony is defined as contractile timing dyssynchrony between the various left ventricular walls, in particular, the septal wall and the lateral wall. The intraventricular dyssynchrony can readily be measured by creating an electric field between two relatively unmoving electrodes (e.g. pacemaker can and electrode in basal region of heart) and measuring sensed voltage changes (e.g., resulting from contractile motion) in a sense electrode attached to the septal wall and a sense electrode in the left ventricle lateral wall (referenced to another electrode which may or may not be one of the driving electrodes), e.g., using the devices and systems reviewed above, such as the electrode configuration is described below and shown in FIG. 1. The intraventricular dyssynchrony is calculated by measuring the time interval between the contractile motion of the sensing elements in the septal and lateral walls. Several time stamps of the contractile motion, such as onset of systolic contraction, peak systolic contraction, and peak velocity of contraction, can be used to make this calculation.

Interventricular mechanical dyssynchrony is defined as the global timing dyssynchrony between the right and left ventricle. The interventricular dyssynchrony can be determined by generating a continuous, e.g., an electric field, between one unmoving electrode (e.g. pacemaker can) and the septal wall sensing element, e.g., electrode, and measuring sensed voltage changes (i.e. contractile notion) in sense electrodes attached to the right and left ventricle lateral walls. These electrode positions are shown in FIGS. 1, 3, 4, 5, 6. Observing sensed voltage changes of the left and right ventricle lateral wall sense electrodes provides global contractile timing information of the left and right ventricles. The interventricular dyssynchrony can be calculated by measuring the time interval between the global contractile motion of the right and left ventricle electrodes.

Another embodiment of the inventive device includes the use of epicardial cardiac leads or multi-electrode patches which are secured to the outside surface of the heart, such as those described in pending U.S. Provisional Application 60/706,641; the disclosures of which are herein incorporated by reference. In this case, the electrodes can be used in the same way for cardiac wall motion sensing, e.g., for CRT optimization, as the right ventricle endocardial leads and the left ventricle cardiac vein leads that are described above.

In certain embodiments, the subject invention provides electrode guidewires for CRT. In these embodiments, a guidewire with one or more electrodes that is used not just for navigation but for CRT optimization during implantation of CRT leads and pacemaker is provided. The guidewire is placed in desired left ventricle (LV) cardiac veins and the electrode is electrically coupled with other electrodes placed in the heart (e.g. right ventricle (RV) septum, pacemaker can, etc.) to measure motion between the electrodes. After the guidewire has been used to locate the optimal LV pacing site for CRT, an electrode lead is slid over the guidewire and positioned such that the lead electrode matches the location of the guidewire electrode. The guidewire is then removed. There are numerous ways to construct such a guidewire. One construction involves using a standard guidewire construction with a tapered core mandrel attached to a coil at the distal end. The mandrel and coil are coated with an electrical insulation coating such as ETFE. The insulation coating is then removed from a defined segment of the coil to make an electrode. On the proximal end of the guidewire is an electrical connector which connects to an external pacemaker.

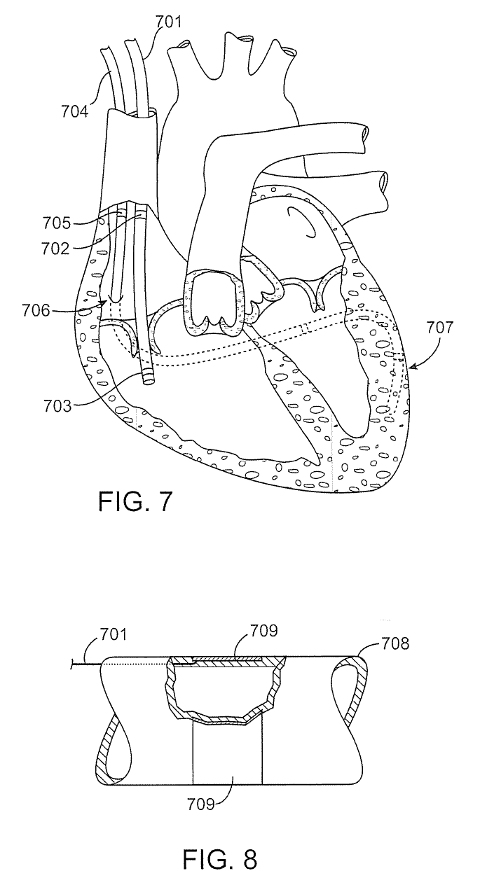

In representative embodiments, the electrode guidewire is constructed as a traditional guidewire and includes an electrode near the distal tip which is used as part of the cardiac wall motion sensing system. This device is used acutely during the placement of the CRT permanent pacing leads. The electrode guidewire is used to determine the optimal placement of the left ventricle electrode leads by placing the electrode guidewire in various locations of the left ventricle cardiac venous system and testing the CRT by alternating the use of the electrode on the guidewire as a pacing electrode and a motion sensing electrode. As such, in certain embodiments, electrode guide catheters or analogous devices are employed to determine optimal or correct positioning of leads for practice of electrical tomography applications of the invention. FIGS. 7 and 8 depict an electrode guide catheter 701 which can also be in the form of an introducer, sheath, sleeve or other catheter type component of a catheter delivery system. FIG. 7 shows the guide catheter 701 which has-been placed into the right ventricle. The guide catheter 701 is embedded with one or more electrodes 702, 703 along its length. There is also a second electrode guide catheter 704 which has been placed through the coronary sinus 706 and into the cardiac vein 707. Also embedded along guide catheter 704 are one or more electrodes.

The guide catheters 701 and 704 are used as part of the delivery system for cardiac leads in the right ventricle or in the right atrium as well as in the coronary sinus and cardiac vein. During such procedures, it is advantageous for the clinician to monitor regional timing and magnitude of cardiac contractions along guide catheters 701 and 704 in the right atrium, the coronary sinus, the cardiac vein, and the right ventricle. The proximal electrodes on the electrode guide catheters 702, 705 could also be used as fixed reference points when they are located in a basal portion of the heart which is essentially not moving.

The main construction of an electrode guide catheter can be accomplished using well known techniques for guide catheters. Such standard fabrication methods typically involve a triple layer construction 708, as shown in FIG. 8. Typically provided is a PTFE liner on the inside surface. In this design, a non-conductive braid wire is placed over the PTFE liner. Over this construct, a nylon or other plastic material sleeve is thermoformed into place with an electrode on the outside layer. The electrode 709 would typically be provided one wire 710 which connects to an electrical connector on the proximal end of the guide catheters 701 or 704.

The guide catheter which is placed into the coronary sinus can also include a smaller electrode guide catheter which can then be advanced much further beyond the coronary sinus and into one of the cardiac veins. This allows measurements of timing and motion of regional contractions near the cardiac vein and left side of the heart. Such measurement would ideally be provided by electrodes situated along the lateral wall of the left ventricle. During the delivery of the cardiac leads, the electrode guide catheters are used for measuring interventricular and intraventricular dyssynchrony and thereby used to optimize cardiac resynchronization therapy variables such as location of pacing leads and pacing timing parameters such as AV and VV delay.

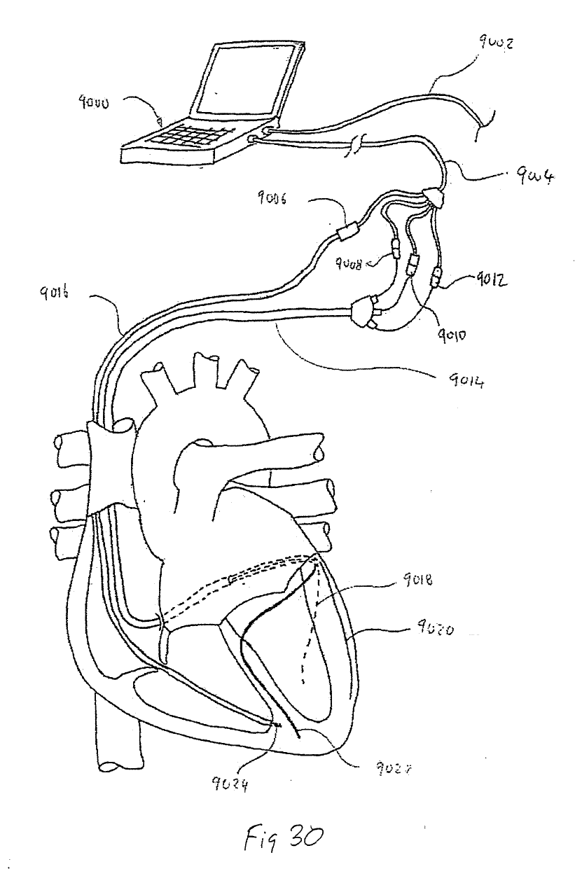

In certain embodiments, the invention provides a quick and easy method to obtain real time information that allows the physician to select the best cardiac vein for optimal CRT. An example of such an embodiment shown in FIG. 30. The device shown in FIG. 30 could use any of the continuous field methods to measure tissue motion described in this patent application. However, for ease of description, the representative embodiment depicted in FIG. 30 uses the electrical tomography technique to measure dyssynchronous cardiac motion and assist in optimizing cardiac resynchronization therapy (CRT) for congestive heart failure (CHF) patients as described in this patent application.

In FIG. 30, the device is comprised of an electrical tomography system 9000 with hardware and software for generation of electrical fields, cardiac pacing, data acquisition, data processing, and data display; a skin electrode cable 9002 is connected to three pairs skin electrodes (right/left torso, chest/back, and neck/leg) which are used to generate three orthogonal electrical fields across the heart; a cardiac electrode cable 9004 which is connected to the internal electrodes within the heart; a guide catheter 9014 which is inserted into the subclavian vein and used to access the coronary sinus; one or more multielectrode guidewires/minicatheters 9018, 9022, and 9024 which have multiple electrodes at the distal end and are inserted via the guide catheter 9014 into the main cardiac vein and its side branches such as the lateral and posterior-lateral cardiac veins; and a standard RV lead 9024 with an active fixation helical electrode 9024 attached to the septal wall.

One embodiment of procedural steps is as follows. The three pairs of skin electrodes are placed on the patient to create the three orthogonal electrical fields spanning the heart. The skin electrode cable 9002 is used to connect the skin electrodes to the electrical tomography system 9000. Under sterile field the physician inserts via the subclavian vein an RV lead into the right ventricle and screws the active fixation helical electrode into the septal wall. The physician then uses the guide catheter 9014 to cannulate the coronary sinus. A venogram using a balloon catheter inserted through the guide catheter 9014 is performed to map the cardiac vein anatomy. The multielectrode guidewires 9018, 9020, 9022 are inserted into the guide catheter 9016. The first multielectrode guidewire 9022 is advanced into the great cardiac vein along the septum until it reached the apex of the heart. This multielectrode can in addition to the RV electrode lead be used to track the motion of the septal wall. The second multielectrode guidewire 9020 is steered into one of the lateral cardiac veins of the left ventricle. And the third multielectrode guidewire 9018 is steered into one of the posterior-lateral cardiac veins of the left ventricle. The cardiac cable 9004 is plugged into the electrical tomography system 9000 and connected to the proximal connectors 9008, 9010, 9012 of the multielectrode guidewires 9018, 9020, 9022, and the proximal IS-1 connector 9006 of the RV electrode lead 9016.

Once all the devices are in place and connected, the three orthogonal electrical fields are turned on and a baseline measurement of the measured motion of all the electrodes is recorded. The amount of baseline intraventricular dyssynchrony is calculated by comparing the motion of the electrodes in the lateral and postero-lateral cardiac veins (multielectrode guidewire 9018, 9020) and the electrodes along the septum (RV lead distal electrode 9024 and/or multielectrode guidewire 9022). Next, CRT test is initiated by performing biventricular pacing with the RV lead distal electrode 9024 and one of the LV electrodes in the lateral or postero-lateral cardiac veins (multielectrode guidewire 9018, 9020). Biventricular pacing is repeated with each of the LV electrodes one by one (multielectrode guidewire 9018, 9020) while recording the corresponding intraventricular dyssynchrony indices. It is important to note that while the LV pacing location is being changed with each test, the motion sensing electrodes used to measure the intraventricular dyssynchrony are not changing position relative to the heart. This allows direct comparison of intraventricular dyssynchrony measurements between all the tests. The data from all the tests is used to generate a map of the optimal LV pacing sites for CRT, thereby identifying the best cardiac vein for placement of the LV electrode lead.

At this point the multielectrode guidewire which is located in the selected cardiac vein is left in place while all the other ones are pulled out. The proximal connector 9008, 9010, or 9012 of the multielectrode lead left in place, is removed and the implantable LV electrode is inserted over-the-wire into the selected cardiac vein and positioned under fluoroscopy to match the position of the determined ideal LV pacing site. In the case of implantation of the multielectrode Protoplex lead, position within the selected cardiac vein is not critical because of the flexibility provided by the multiple electrodes along the lead.

In another embodiment, at this point all of the multielectrode guidewires are removed and under fluoroscopy the LV electrode lead is positioned using standard lead delivery tools to match the position of the most ideal accessible LV pacing site. Finally, the standard CRT implantation procedure is resumed.

In summary, this inventive device offers the physician a quick and easy tool to generate a clear map of which cardiac veins provide the best LV pacing sites for optimal CRT, and thereby this invention answers to the currently unanswered question of where to place the LV lead during a CRT implantation procedure.

Other embodiments of the inventive electrode guide catheter include an electrode introducer, electrode sheath, or electrode sleeve, all of which can make part of the delivery system of cardiac leads. One advantage of these configurations is the simplicity of integrating electrodes into these catheter type devices. Another advantage is that these electrode catheters are compatible with already existing implantable cardiac pacing systems.

In certain embodiments, the transmit and receive signals are coupled to the intracardiac leads using a non-contact method such as inductive coupling. For instance, a coil placed around the lead and electrically connected to-the transmitting signal source could couple the RF signal onto the lead without any physical contact between the lead and the signal generator.

In certain embodiments, the systems and methods are employed to measure coupling between other electrode locations. The placement and selection of electrode pairs will determine the physical phenomenon that is measured. For instance the voltage coupling between an electrode in the right ventricle and an electrode in the right atrium provides an indication of the timing of the tricuspid valve closing and opening. In certain embodiments, a multiplicity of electrodes on a single lead. For instance a LV pacing lead may have electrodes in addition to the conventional pacing electrodes that extend from the vena cava, through the coronary sinus, and into a cardiac vein on the LV freewall. By selecting different pairs of these electrodes, different aspects of the heart's motion may be measured, as desired.

In certain embodiments, electrodes are placed in the guide catheters and/or guide wires that are used in various procedures, e.g., placement of a lead in the coronary sinus for CRT, and the electrical signal received from them gives the physician additional information about the location of the catheters or wires during the procedure, which aids navigation. For instance if the transmitting electrode was in the RV and the receiving electrode was on the tip of the guide catheter, the physician will observe a large change in signal magnitude when the guide catheter crosses the tricuspid valve. Since the entrance to the coronary sinus is very close to the tricuspid valve (which is not visible under fluoroscopy), such an observation provides useful information. Further changes in signal are observed when the catheter entered the coronary sinus, and may be used for detection of such.

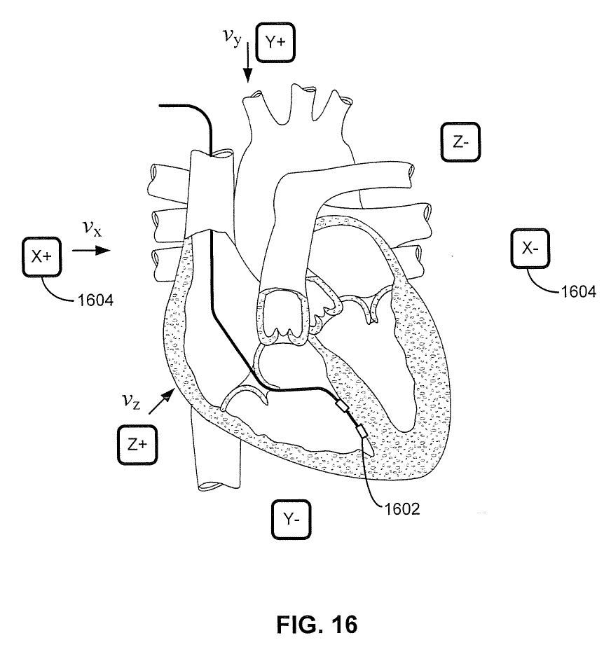

In certain embodiments, a plurality of drive electrode pairs are present, each generating a distinct electric field, where the fields are generally oriented along different endocardial planes, e.g., as may be generated by the different driving electrode pairs shown in FIG. 16. Representative planes generated in certain embodiments are between relatively immobile electrodes located in the superior vena cava, the coronary sinus and an implantable pulse generator in the left or right subclavicular region. Additional electrode locations include the pulmonary artery, and subcutaneous locations throughout the thorax, neck and abdomen, as well as external locations.

In certain embodiments, additional planes are generated from electrodes experiencing relatively greater motion than those already described (e.g., right ventricular apex, cardiac vein overlying left ventricle, etc.). In representative embodiments, to obtain absolute position, computational techniques are employed with reference to other available planes in order to eliminate the motion component of the drive electrodes with respect to the sense electrodes. In certain applications of the system, relative timing and motion information is of greater importance than absolute position. In these applications, at least, significant movement of one or more electrical field planes may be tolerated with minimal or even no real-time computation intended to compensate for this motion.

In certain embodiments, detection systems currently available for monitoring movement of a catheter inside a body are adapted for use in the subject methods. Representative such systems include the LOCALISA® system from Medtronic, Inc., as described in U.S. Pat. No. 5,983,126 (the disclosure of which is herein incorporated by reference) and the ENSITE NAVX™ system from St. Jude Medical, e.g., as described in U.S. Pat. No. 5,662,108, the disclosure of which is herein incorporated by reference. These systems incorporate skin patch electrodes transmitting a small alternating transcutaneous current to generate electrical fields. The amplitude of each frequency component recorded at each intracardiac recording site is used to resolve position in three dimensions. Of note is that both of these inventions are intended to reduce patient exposure to ionizing radiation during lengthy catheter ablation procedures. Since the intent is solely to localize roving intracardiac catheters, these systems are specifically designed so that cardiac wall motion—the parameter captured in the present invention—is not recorded. Means of cardiac motion elimination include, narrow bandwidth of the delivered alternating current signals, gating data acquisition to the cardiac cycle, and averaging the delivered data over lengthy (i.e., one to two second) time intervals.

These systems are readily modified in order to track cardiac motion in accordance with the present invention. In order to do so, these systems are adapted to provide at least temporary if not permanent fixation of recording (i.e., sensing) electrodes in association with the region of the heart to be monitored. In addition, delivered alternating current frequencies are sufficiently separated to permit the higher bandwidth data capture desired to accurately and precisely characterize cardiac motion within the cardiac cycle. In addition, cardiac cycle gating and signal averaging techniques are adapted to permit acquisition of clinically meaningful intra-cardiac-cycle wall motion data.

In one embodiment of the present invention, skin patch electrodes are provided, with the modifications just described, in order to derive acute wall motion information. In another embodiment, an implantable cardiac rhythm management device, such as a pacemaker, or an implantable cardiac performance monitoring device is equipped with a “clinic mode” whereby intracardiac electrodes provide position amplitude data from externally applied electrical fields. In this regard, important cardiac performance parameters may be non-invasively recorded at the time of a physician visit, or even at home on a temporary basis, under both resting and exercise conditions. In a further embodiment, the system just described includes the intracardiac field generation capability described earlier, but incorporates the ability to also recognize additional, temporarily applied electrical fields. In this embodiment, for example, a cardiac resynchronization pacemaker reports data used by the physician to select optimal left and/or right ventricular stimulation location(s) using multi-electrode endocardial and/or epicardial leads. In certain embodiments, the system self-optimizes by operating in a closed-loop fashion to ensure optimal cardiac synchrony. The system of this example or another cardiac monitoring system employing endocardial electrical field plane(s), as previously described, also incorporates a “clinic mode” in certain embodiments, whereby the application of external electrical fields enhances the resolution of the entire system. This additional resolution proves useful in providing clinically useful quantitative cardiac performance parameters or in calibrating the permanently implantable components of the system.

In yet other embodiments, an electrode bending sensor for CRT is provided. These embodiments exploit the use of a pair of electrodes on a single lead as a bending sensor. In one embodiment, electrodes in close proximity (e.g. 1 cm apart) are electrically coupled. When the lead is bent, the distance between the electrodes decreases thereby changing the electrical coupling. The measured electrical coupling signal provides regional timing and magnitude information related to bending of the lead in the cardiac region around the electrodes. The comparison of multiple electrode bending sensors placed throughout the heart can be used to obtain mechanical dyssynchrony data, e.g., for CRT optimization.

### Electrical Synchrony Measurement of Cardiac Function

One representative embodiment of the electrotomographic embodiments of present invention is an electrical synchrony approach, as reviewed below. This representative method allows for the first time an electrical synchrony measurement. This embodiment of the present invention also measures wall motion. However, with this embodiment of the present invention, wall motion measurement is not required for synchrony measurement.

In this embodiment of the present invention, a number of electrodes are provided on a cardiac lead. Electrodes placed for other purposes can also be employed in this system. In a representative embodiment, these electrodes are identified as E0, E1, E2, E3 etc, which electrodes could be located at various places of interest, e.g., in the LV. Additionally, an electrode, EC, may be provided which would be in the right ventricle, with an electrode, ED which is located in the right atrium. In addition, the pacemaking can is employed in this embodiment of the present invention as a separate electrode. Accordingly, the pacemaking can is susceptible to utilization as an ‘electrode’ to contribute to the information generated by the inventive system. Where desired, an array of additional electrodes, here designated as E′ may also be included in the present embodiment of the inventive system. By example, these electrodes can be located subcutaneously around the heart. This system would also include the pacemaking can as one location for analysis, designated E′₁, with at least one additional electrode E′₃. In the utilization of the inventive system of this representative embodiment, an AC signal is set up between various electrodes. By example, EC would be provided with an AC signal. The corresponding counter electrode in this case could be the pace maker E′₁ or one of the electrodes on that percutaneously placed lead (underneath the skin), which would be the relevant ground.

A lock-in amplifier is then conveniently employed, when desired, to sample the voltage at E0, E1, E2 or E3. In this representative embodiment, the lock-in amplifier measures the voltage, and particularly the DC component of the voltage. By example, one can select E3 and ED for a sensing process. These electrodes are preferably positioned on a more or less straight line with E′₃. A lock-in amplifier is provided which gives the DC potential at E′₃. An important innovation in this example is that this lock-in amplifier is run at two different frequencies, e.g., a first frequency ranging from about 4 KHz to about 20 KHz; and a second frequency ranging from about 25 KHz to about 300 KHz. What allows the production of the resynchronization data is that blood and tissue have different impedances at those different frequencies.

A lock-in amplifier is provided between the relevant electrodes, serving to put the voltage between ED and EC. The return path is to E′₃. As a result, the potential at E3 will be a function of the distance between E3 and ED and E3 and E′₃. The potential will also be a function of the relevant impedances along that line of paths. In this inventive embodiment, there is no sampling of impedance. Rather, the sampling is of potential. There is also no measuring of impedance in any way, but rather voltage is determined.

The potential at E3 will be a function of both the distance between E3 and ED and the composition of the material between E3 and ED. This measurement is significant for clinical insight because the resistance, for example, of the tissue in the septal wall, will be different than the specific resistance or impedance of the blood inside the left ventricular volume. As a result, the two frequencies that are chosen will be selected to have different relative impedances.

In the case described above, at low frequencies (e.g., about 10 MHz), there may be about a 10-300% difference, such as about 50-250% and including about 100-150%, in the blood resistance vs. the tissue resistance. The resistance varies with the frequency. At higher frequencies (e.g., about 1 MHZ) the ratio approaches unity. By example, blood resistance may be at about 160 Ωcm, while-cardiac tissue may vary from about 160-400 Ωcm. The frequencies to employ in a given application will be readily determined by one of ordinary skill in the art through standard experimentation, or review of the literature.

In the above described representative embodiment, the potential at E3 will change not just because of volume. The potential will change if different sample frequencies are employed. The different numbers that are obtained between the two media of transmission allow the determination of the percentage of the ratio of tissue and blood between E3 and ED.

When the heart contracts, the cardiac wall becomes bigger in its cross-sectional dimension. As the wall gets ‘bigger’, the outside dimensions change to some degree. At this point, the distance of tissue is in flux. LV thickness is modified during systole, as is septal thickness. The dimensions of the LV blood area at systole is also modified. As a result, the LV thickness distally is much greater than the same dimensions in systole. One could also make the analysis of the LV systole divided by the sum of the septal thickness systole plus the LV systole plus the LV thickness systole.

Using the above knowledge, one can readily determine with this embodiment a parameter of heart function referred to herein as the blood tissue ratio, hereinafter the BTR. The BTR equals the distance from the inner wall of the LV septum to the inner wall of the LV outer wall. This value is the ratio of the distance that is blood, divided by the distance between the electrode on the septal wall and the electrode on the outer LV artery. This system provides a measurement for each location which is actually a ratio of cavity length over the sum of the cavity length: both wall thickness.

For each of the various electrodes in the system, e.g., E0, E1, E2, E3, E4, E5 etc., and compared to the points of EC, ED, etc. along the LV wall, there will be a variety of these BTR measurements and synchronies. In this case, the BTR will have a value as a function of time. The BTR can be instantaneously computed with modern computational techniques. This computation is a very simple calculation to accomplish because instead of measuring distance, the actual measurement is of the BTR.

BTR as determined by this representative embodiment of the present invention is a function of time. The measurement provided by the device of the present invention can be displayed as curves of BTR as a function of time for each of the different points being assessed in the system. As the clinician provides effective resynchronization therapy, improved synchronicity may be determined by the point where each of the points is at maximum systole. Where the blood thickness ratio is a minimum, the measurements will line up. That is the point where the amount of blood between the two inner walls is a minimum.

The goal of the clinician seeking to optimize resynchronization therapy using the sensors of the present invention will be to modify the therapy until all of these electrodes and all their BTR measurements are small at the same time.

There are multiple methods well know to the ordinary skilled artisan of measuring to determine when two numerical associations are small at the same time. By example, the determination of a time between when the QRS interval begins and the point of BTR minimum for each or the electrode pairs used in the measurement. All of those different times are noted, for example, and a standard deviation of variation of, say, 12 different segments are computed. As a result, the standard deviation of these is twelve times the synchrony measurement.

### Electrical Doppler Tomographv Embodiments

As reviewed above, another continuous field property that can be monitored by a sensing element in the subject tomographic applications is frequency of a continuous signal as perceived at a sensing element. These embodiments are also referred to herein as Doppler embodiments.

In representative “Doppler” embodiments of the present invention, the term “Doppler transmitter/sensors” refers to a range of implantable features, that may be transmitters only, may be sensors only, or may have the capacity to serve both as a Doppler transmitter and sensor, either at alternate times or simultaneously. Included within this meaning is the use of existing electrodes or other cardiac elements which can serve in this capacity in the context of the overall inventive system. Thus, current available and/or implanted pacing or sensing electrodes can serve as Doppler transmitter/sensors within the inventive system even if they were not initially designed or implanted to serve in that capacity.

The Doppler tomography method of these embodiments of the present invention can be provided much in a manner analogous to ultrasound used in the clinical environment. Additional methods used in radar and in other applications for tracking the speed and position of everything from aircraft to automobiles to baseballs can be used in the present inventive methods.

By employing a variety of electrode pairs in the present Doppler tomography system, each broadcasting in a discrete frequency, multiple lines of position and velocity can be calculated from differing reference frames. This embodiment of the present invention creates a Doppler tomogram providing an enormous amount of clinically relevant velocity and positional information in real-time. As a major advancement over currently available clinical ultrasound methods, these data provided by the inventive Doppler tomography system would be inherently machine-useable as the positioning velocity data are numeric rather than an image requiring human interpretation with all the inconsistencies inherent in individual interpretation.

A further advantage of the inventive Doppler tomography system of these representative embodiments is that the influence of reflected signals from regions far from the area of interest is reduced. That is because the inventive system does not rely on reflected signals. Rather, the present system is informed by directly transmitting signals to a receiving electrode and/or electrodes located elsewhere in the heart, the body, or on the surface of the skin.

The present invention can be implemented in the practical deployment of multiple sensors to describe in further detail wall motion on a segmental basis.

Accordingly, the inventive Doppler tomography system of these representative embodiments of the invention uses electromagnetic energy to determine position of various cardiac structures. Unlike prior sensor approaches to providing data on cardiac wall position, the present Doppler tomography system determines these positions by exploiting the Doppler frequency shift caused by relative motion of the cardiac walls with respect to various electrode pairs located intra or extracardiac.

One advantage of the inventive Doppler tomography techniques is that direct position information can be calculated by a single integration of the Doppler signal. This unique quality is in contrast to such sensor approaches as accelerometry which require double integration. A further advantage of the inventive Doppler tomography system is that direct relative velocity, which can be very valuable in optimizing biventricular pacing, is immediately available from the Doppler signal or signals themselves.

The Doppler tomography method of the present invention is in some ways similar to ultrasound used in the clinical environment. However, by employing a variety of electrode pairs in the present Doppler tomography system, each broadcasting in a discrete frequency, multiple lines of position and velocity can be calculated from differing reference frames. Thus, a Doppler tomogram is created. This unique data providing, for the first time, clinically relevant velocity and positional information in real time. This data is inherently machine-useable as the positioning velocity data are numeric rather than an image requiring human interpretation with all the inconsistencies inherent in individual interpretation. The present system is informed by directly transmitting signals to a receiving electrode located elsewhere in the heart.

The central principle being used by the present inventive Doppler tomography system of these representative embodiments is to obtain positional and velocity information using the Doppler shift. This phenomena has been well characterized and applied to all forms of electromagnetic radiation as well as acoustic radiation. The standard formula states the change in wavelength observed due to relative motion equals the wavelength first injected into the system multiplied by the velocity vector directly towards the transmitter and or receiving system divided by the conduction velocity of the waveform in the material of interest. For example, in the case of radar guns used in the air, that speed would be approximately the speed of light. This principle in the present invention is applied radio waves that are transmitted by the inventive Doppler transmission/sensor units.

In a representative embodiment of the current invention, the conduction velocity is via ionic conductance of an applied RF signal in the body. Consistent with data developed by the present inventor, this conductance velocity is approximately 10% to 15% the speed of light in physiologic normal saline.

Other embodiments of the present invention employ sufficiently high frequencies and small antennae designs embedded in the intracardiac catheter that a light speed radiated signal is used. Other embodiments include ultrasound transducers for converting the applied electrical signal into acoustic energy. In this case, the acoustic energy is then received by the receiving transducer. The signal is then recorded in that means and using the speed of sound in the human body as the conduction velocity, the relevant information calculated using the Doppler formula.

In a representative embodiment of the present invention, radio frequency energy is delivered at low power and transmitted via conductance. Each emitting electrode pair is also potentially a receiver. As a result, each pair of electrodes is capable of both broadcasting a continuous field, and can also either simultaneously or at a different time sense the field from the various other transmitting electrodes. The frequency bands are sufficiently separated such that the received frequency shift could be accurately recorded and its source determined.

In additional embodiments of the present invention, computation ornaments are added to the system even on an implantable basis for full time analysis or via download or real time interrogation on an external basis in order to compute the parameters of interest at any given time.

Doppler shift has not yet been reported or used in the context of an implantable cardiac device. The current invention offers both a solid state and constructible, reliable means of optimizing biventricular pacing both in terms of location and timing. This allows prompt detection of reversible and irreversible ischemia, especially so-called “silent ischemia”. The invention allows a determination of important hemodynamic parameters on a permanent implantable basis. Such hemodynamic parameters can include such components as stroke volume, ejection fraction, cardiac output and others, as well arrhythmia detection and classification via reliable mechanical means.

The manufacture of the inventive Doppler transmitter/sensor point has particular advantages over other sensors. Active devices such as accelerometers can be difficult to fabricate. This difficulty is particularly accentuated in the very small sizes required for incorporation into implantable leads or other means of intracardiac implantation. Furthermore, hermetically sealing such devices from the corrosive environment of the body is problematic. Additionally, delivering power and data in reliable fashion to such sensors adds to the challenge of producing a highly robust system.

A benefit of the current inventive Doppler tomography system is that conventional intracardiac electrodes can be used. In fact, electrodes used in the inventive system may be the same electrodes used for other purposes. By example, electrodes used in cardiac sensing of ECG, cardiac pacing and delivery of defibrillation pulses can be employed. Since these other activities of the electrodes occur on significantly different frequencies from the Doppler methodology of the present invention, no interference would occur between the multiple purposes to which such electrodes could be used.

If ionic conduction velocity is selected in an embodiment of the present invention rather than free spatial electromagnetic radiation, a calibration of conduction velocity may, in some instances, be required. One approach to these challenges is to time a transmission crossing distance such as the distance between electrode pairs on an implantable device such as an implantable lead. If ionic conduction velocity were found to vary significantly between blood and tissue, correction factors can be incorporated in order to reduce the noise inherent in the data. Alternatively, this factor could be omitted if such conduction velocity differences were not significant as compared to the signal itself.

The devices of the present invention may be fabricated to utilize frequencies in the acoustic domain such as ultrasound transducers or small antennae utilizing free space radiation in a very high frequency domain. In the case that multipath signals caused by multiple reflections are a limiting factor, processing, power and selective filtering would ameliorate these effects. Therefore a preferred embodiment of the inventive Doppler tomography system is to use the lower frequencies associated with ionic conduction in order to simplify the initial application of the invention.

One important distinguishing feature of these embodiments of the present invention is that, unlike radar or external beam ultrasound, the current invention does not rely upon reflected energy returning to the emitter in order to acquire data. Instead, the invention relies upon primary emissions from electrode pairs or other transducers being received by transducers in a receiving mode located at another location.

Using the devices and methods of the present invention, the timing and displacement of contraction of the monitored sections of the heart can be compared to one another, phase and amplitude differences evaluated, and means manually or automatically taken to move contraction of wall segments into synchronization with one another. In this way, the maximum contraction occurs at essentially the same time or the time most efficient from the standpoint of producing the greatest hemodynamic output for the least amount of effort.

In one embodiment of the present invention, resynchronization data is obtained by means of localizing endocardial elements along the right ventricular septum and an aspect of the left ventricle. This can be accomplished, either by the endocardial approach through a cardiac vein, or through an epicardial approach analogous to placement of an epicardial left ventricular stimulation electrode. The inventive device in this case is configured to describe the relative position of the different wall segments relative to one another.

A representative embodiment of this approach involves the placement of one or more Doppler transmitters/sensors along a lead located in close association with the right ventricular septum, and also in addition, a lead located in a cardiac vein located on the left ventricular surface. An alternative would include a lead using Doppler transmitter/sensors placed in the antero-septal vein that roughly tracks the inter ventricular septum and another further laterally or posteriorly along the left ventricular surface.

In another aspect of the present invention, additional Doppler transmitter/sensors are placed along the aspect of the right ventricular free wall. This provides an understanding of interventricular dissynchrony, rather than intraventricular dysynchrony within the left ventricle itself. These data are particularly useful in cases of both right ventricular heart failure and right sided heart failure.

A representative embodiment of the present invention is configured as an implantable system with either a can, hermetically sealed can with a battery and processing gear, or a coil designed for subcutaneous placement. With this inventive configuration, power and data can be transmitted through the skin to the device. Two leads extend from the inventive device. One of these leads is placed in the right ventricle in close association with the interventricular septum. The second lead is positioned to access the coronary sinus by being placed along another aspect of the left ventricle through a cardiac vein. Alternatively, the leads can be positioned in a manner analogous to the cardiac resynchronization therapy process. For instance, a left ventricular lead could be placed epicardially if suitable cardiac veins are not available for cannulation.

The system can be configured with Doppler shift sensors along each lead or an alternative position detector, such as a radio frequency or tuned circuit or Hall effect or time of flight sensor, such that the relative position of the sensors one from another can be determined throughout the course of the cardiac cycle.

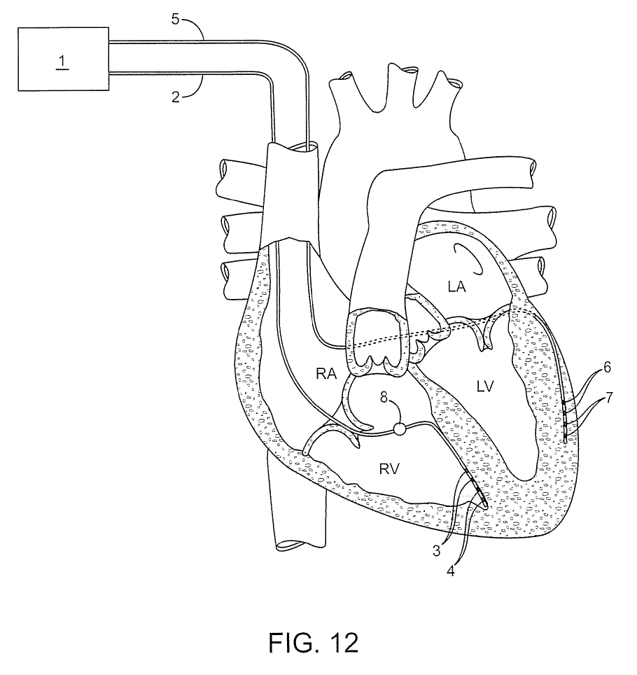

FIG. 12 provides a diagrammatic view of a representative embodiment of the inventive implantable Doppler tomography system. Communication element 1 provides the extracardiac communication and calculation element for the overall system. Communication element 1 can take the form of various embodiments including an implantable device complete with power supply, drive electronics and processing power on board. In more complex configurations, communication element I provides a means for communicating data and power from a completely external or extracorporeal location.

Right ventricular lead 2 emerges from communication device in communication element 1, and travels from the subcutaneous location of communication means 1 via the subclavian venous access through the superior vena cava through the right atrium and then through the tricuspid valve to a position along the right ventricle. This location is located along its distal portion in close association with the intraventricular septum terminating distally with fixation in the right ventricular apex.

Particular to distal aspect of right ventricular lead 2 are right ventricular electrode pairs 3 and 4. In other embodiments of the present invention, an additional number or smaller number of electrodes may be employed.

Additionally emerging at the proximal aspect of communication element 1 is left ventricular lead 5. Left ventricular lead 5 starts by following the same route as right ventricular lead 2 via subclavian vein through the superior vena cava into the right atrium. At this point, left ventricular lead 5 is placed via the coronary sinus around the posterior aspect of the heart and thence into cardiac vein draining into said sinus.

FIG. 12 further depicts left ventricular lead 5 in a position likely to be advantageous for biventricular pacing located along the lateral aspect of the left ventricle. Left ventricular electrode pairs 6 and 7 are shown in this drawing analogous to electrode pairs three and four which are previously described.

Right ventricular lead 2 may optionally be provided with pressure sensor 8 which is located in the right ventricle. Pressure sensor 8 provides a pressure signal which can also simultaneously be obtained with wall motion data. It is notable that adding active devices to said lead such as pressure sensor 8 is facilitated through use of a multiplexing system, which has been previously disclosed and may or may not be used in this case.

Principle of operation of the inventive implantable Doppler tomography system is that a communication element 1 will either communicate or generate a radio frequency at different frequencies. By, example a 30 kilohertz signal can be provided with a 100 or 200 kilohertz shift for each successive electrode pair. The frequency perceived at left ventricular electrode pairs 6 and 7 would be routed back to communication element 1 and the originating frequency subtracted from the received frequency using the mixer. The resulting frequency would represent the frequency shift and that could be used via the Doppler formula to calculate the instantaneous velocity. Processing of this data could also resolve position by integration. Performing the first derivative of this data could also yield acceleration information.

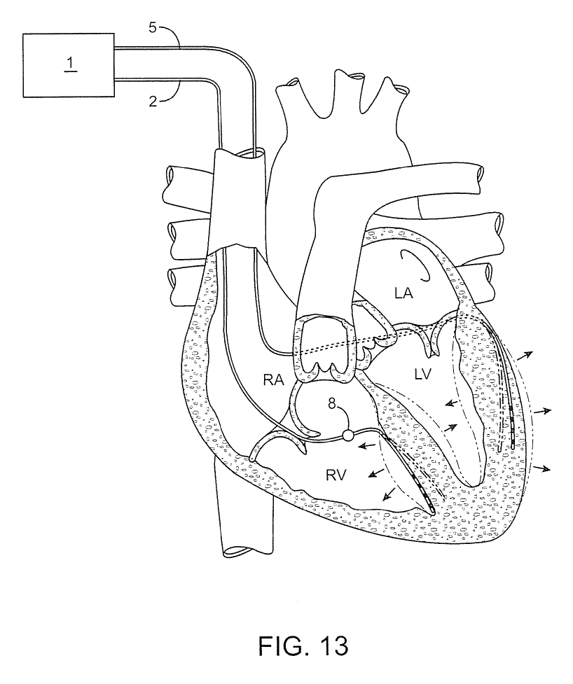

FIG. 13 depicts the roles of the heart in motion. With a lead such as right ventricular lead 2 and left ventricular lead 5 in close association with the wall of the heart as the wall of the heart moved via 3D cardiac cycle and so would the catheters in a proportionate amount. As these catheters moved towards and away from one another, the range and velocity information derived from the aforementioned method would shift over the course of the cardiac cycle in a manner indicative of their movement and timing of said movement.

The position data and extent of the Doppler shift together with an optional pressure signal or signals is used, for example, to optimize cardiac resynchronization therapy where the goal is to maximize the contractility of the left ventricle. This is obtained by encouraging effectively simultaneous contraction of the bulk of the muscle of the left ventricle.

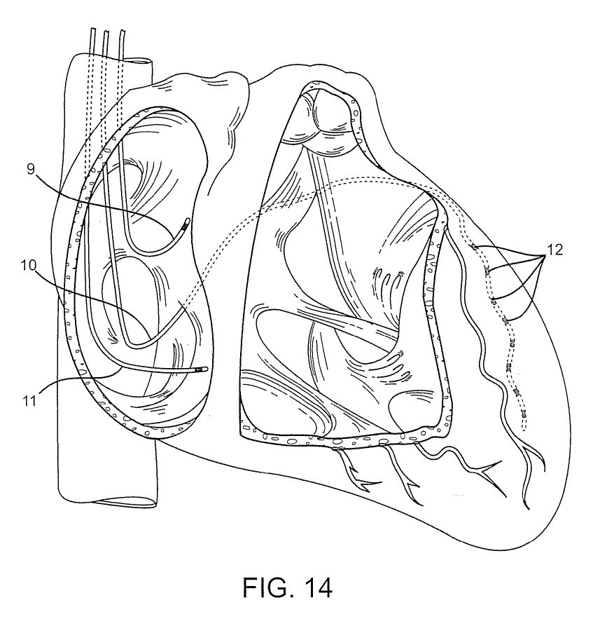

FIG. 14 shows the posterior aspect of the heart. In this case, three leads are depicted which would be the typical state in a biventricular pacing system in which the current invention could be integrated in another preferred embodiment.

Depicted graphically in right atrial lead 9 is a right atrial pacing lead. A left ventricular lead 10 is depicted entering the coronary sinus and then the dash lines indicting passage through the coronary sinus and thence along a cardiac via the interior of a cardiac vein along the left ventricular surface. Right ventricular lead 11, while not shown the current view, is preferential positioned intimately along the intraventricular septum.

By means of VCR and the various electrodes 12 along the left ventricular lead 10, each of these could be used potentially for pacing as well as for Doppler shift related position and velocity information according to the manner just described. This information can be taken relative one to another to give a sense for local left ventricular shortening as well as relative to the electrodes located in the right atrium and right ventricle. The additional electrodes can be placed at the subcutaneous implantation site of an implantable generator or coil.

### Additional Electrical Tomography Embodiments

One embodiment of the present invention provides a system for locating implanted electrodes for cardiac resynchronization. During operation, the system applies a field to a tissue region in which one or more target devices reside. The system then detects a signal from the target device which is induced by the field. Next, the system determines a displacement or a movement of the target device based on the detected signal and characteristics of the applied field.

A further embodiment of the present invention provides a system for determining displacement of a target electrode implanted in organic tissues. During operation, the system facilitates two driving electrodes coupled to a tissue region. The system also facilitates an auxiliary electrode in the vicinity of each driving electrode and facilitates two operational amplifiers. One input of each operational amplifier is coupled to one auxiliary electrode, and the output of each operational amplifier is coupled to the driving electrode which is in the vicinity of the auxiliary electrode coupled to the operational amplifier's input. The other input of each operational amplifier is coupled to an AC voltage source. The system then measures an induced voltage on the target electrode and determines an approximate displacement of the target electrode based on the induced voltage.

Another embodiment of the present invention provides a system for determining displacement of multiple implanted target electrodes coupled to a single lead. During operation, the system applies an AC voltage to a tissue region where the target electrodes reside. The system then receives at a target electrode a reference signal with a frequency substantially the same as a frequency of the AC voltage. Next, the system mixes the reference signal with a voltage induced on the target electrode to obtain a mixed signal. The system also filters the mixed signal to obtain a filtered signal and modulates a carrier signal with the filtered signal to obtain a modulated signal, wherein a frequency of the carrier signal is different from the frequency of the AC voltage. The system then transmits the modulated signal.

Another embodiment of the present invention provides a system for analyzing cardiac motion. During operation, the system places n cardiac electrodes and applies an AC voltage to a tissue region where the cardiac electrodes reside. The system then detects an induced voltage on each electrode and constructs a n×n correlation matrix based on the induced voltage on each cardiac electrode. The system subsequently diagonalizes the correlation matrix, thereby solving for eigenvalues and eigenvectors of the correlation matrix.

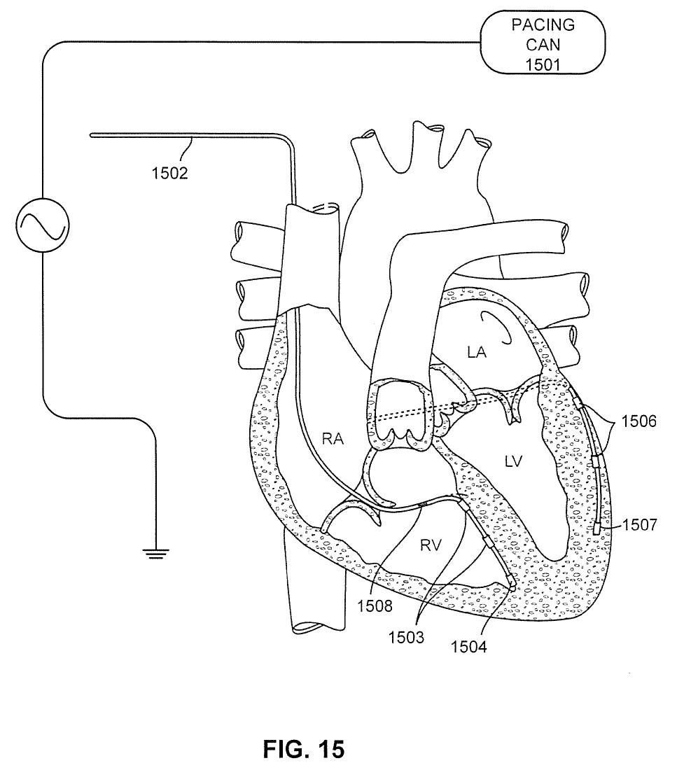

FIG. 15 illustrates an exemplary configuration for electrical tomography of cardiac electrodes, in accordance with an embodiment of the present invention. FIG. 15 shows the locations 1503, 1504, 1506 and 1507 of a number of pacing electrodes. A pacing can 1501 resides in an external or extra-corporeal location. Pacing can 1501 may transmit pacing pulses to the electrodes through a pacing lead 1502.

Electrodes at locations 1503 and 1504 are coupled to right ventricular lead 1502, which travels from a subcutaneous location for a pacing system (such as pacing can 1501) into the patient's body (e.g., preferably, a subclavian venous access), and through the superior vena cava into the right atrium. From the right atrium, right ventricular lead 1502 is threaded through the tricuspid valve to a location along the walls of the right ventricle. The distal portion of right ventricular lead 1502 is preferably located along the intra-ventricular septum, terminating with fixation in the right ventricular apex. As shown in FIG. 15, right ventricular lead 1502 includes electrodes positioned at locations 1503 and 1504. The number of electrodes in ventricular lead 1502 is not limited, and may be more or less than the number of electrodes shown in FIG. 15.

Similarly, a left ventricular lead follows substantially the same route as right ventricular lead 1502 (e.g., through the subclavian venous access and the superior vena cava into the right atrium). In the right atrium, the left ventricular lead is threaded through the coronary sinus around the posterior wall of the heart in a cardiac vein draining into the coronary sinus. The left ventricular lead is provided laterally along the walls of the left ventricle, which is a likely position to be advantageous for bi-ventricular pacing. FIG. 15 shows electrodes positioned at locations 1506 and 1507 of the left ventricular lead.

Right ventricular lead 1502 may optionally be provided with a pressure sensor 1508 in the right ventricle. A signal multiplexing arrangement facilitates including such active devices (e.g., pressure sensor 1508) to a lead for pacing and signal collection purpose (e.g., right ventricular lead 1502). During operation, pacing can 1501 communicates with each of the satellites at locations 1503, 1504, 1506 and 1507.

According to one embodiment, pacing can 1501 is used as an electrode to apply an AC voltage to the heart tissue. The ground of the AC voltage source may be at another location on the patient's body, for example a patch attached to the patient's skin. Accordingly, there is an AC voltage drop across the hear tissue from pacing can 1501 toward the ground location. An electrode implanted in the heart has an induced electrical potential somewhere between the driving voltage and the ground. By detecting the induced voltage on the electrode, and by comparing the induced voltage with the driving voltage, one can monitor the electrode's location or, if the electrode is moving within the heart, the instant velocity of the electrode.

The system may also apply a direct-current (DC) voltage to the tissue. However, an AC driving voltage is preferable to a DC voltage in representative embodiments, because AC signals are more resistant to noise. Because the induced voltage signal on an electrode has substantially the same frequency as the driving AC voltage does, a lock-in amplifier can be used operating at the same frequency to reduce interferences from noise.

The system may apply the electrical field in various ways. In one embodiment, the system may use a pacing can and an existing implanted electrode, or two existing implanted electrodes to apply the driving voltage. In a further embodiment, the system may apply the driving voltage through two electrical-contact patches attached to the patient's skin.

Based on the same principle, one can apply three AC voltages in three directions (x, y, and z), which are substantially orthogonal to each other, to measure the location of an electrode in a 3-dimensional (3-D) space. FIG. 16 illustrates an exemplary configuration for 3-D electrical tomography of cardiac electrodes, in accordance with an embodiment of the present invention. The system applies an AC voltage vₓ through a pair of electrodes 1604 in the x direction. Similarly, the system applies v_{y} and v_{z} in the y direction and z direction, respectively. vₓ, v_{y}, and v_{z} each operates at a different frequency. As a result, three induced voltages are present on an implanted electrode 1602. Each induced voltage also has a different frequency corresponding to the frequency of the driving voltage in each direction. Therefore, by detecting the three induced voltages using three separate lock-in amplification modules, each of which operating at a different frequency, one can determine the electrode's location in a 3-dimensional space.

### Electrical Gradient Tomography

The electrical gradient embodiment of the present invention has several advantages. Electrical gradient tomography corrects for potential nonlinearity in the system. Electrical gradient tomography may be selected in applications where non-linearity is likely, potentially compromising data outside useful limits for a specific need.

The electrical gradient tomography method measures the AC potential at a location between two different electrodes. AC voltage is employed at both the drive electrode and the receive electrode. The receive electrode is placed in a different position in the body from the drive electrode. In the simplest form of the current tomography invention, the variation in amplitude at the receive electrode is related to the distance between the ground electrode and the drive electrode.

Using electrical gradient tomography, it is possible to estimate with greater accuracy the precise location of the electrodes. This is accomplished by determining the rate of change of the AC signal as a function, of distance in more than one direction. This rate of change is a function of distance as the gradient of the AC potential.

By measuring the gradient of the AC potential, as well as the AC potential at the receive electrode location, both the absolute value and the rate of change of the value is achieved. From this information, more accurate data of the motion of that receive electrode as a function of time is accomplished.

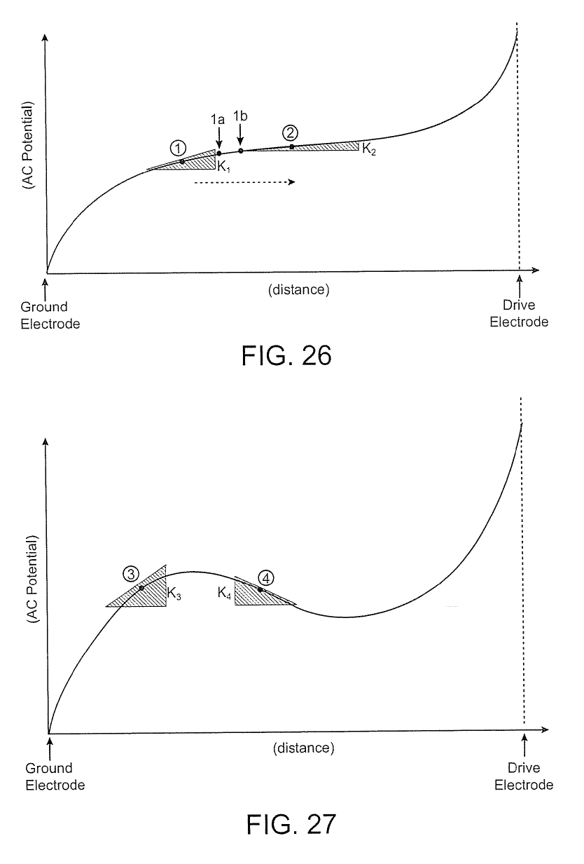

FIG. 26 provides an example of a relatively smoothly operating system among those of the present invention. The AC potential of the receive electrode is plotted as a function of the distance between the ground electrode and receive electrode. From left to right, this plot a monotonic, smooth function. However, the plot is not linear. The plot is grossly nonlinear near the electrodes, that is near the drive electrode and near the ground electrode.

FIG. 27 provides an example of data which can be improved using electrical gradient tomography. As with the prior example, the data to be improved is the potential of the receive electrode as a function of distance between ground electrode and the drive electrode. In this case, however, the potential drops at closer distances to one electrode.

There is an unusual way of analyzing this phenomenon which leads to some of the special advantages of electrical gradient tomography. There are two situations involved. One is where the drive electrode is moving relative to the ground electrode. The other is where the receive electrode is moving sideways relative to the line between the ground electrode and the drive electrode. These situations cause the potential to drop even though the distance between the ground and the drive electrode has not changed.

It is advantageous to calculate an electrode position in three-dimensional space. Using gradient or the slope of the rate of change of the AC signal is an important approach to gaining that position data. As an example of how this approach would be undertaken in one dimension, see FIG. 26. An electrode at location 1 is moving to location 2. As the electrode moves gradually from left to right, the slope of the AC potential as well as the value of the AC potential are recorded.

As the electrode moves somewhat to the right, its distance is measured using the slope and the amplitude. The slope is measured by having closely spaced electrodes that are diametrically opposed in two different dimensions. As the differential voltage is measured across those closest spaced electrodes, the gradient is determined.

As the electrodes move from left to right, their slope and the amplitude are determined. When the electrode moves to the right, the amplitude will change. Based on the slope, the effective distance is computed as the electrode moves from location 1 to location 1a, to location 1b, and eventually the full distance to location 2. The combination of slope and value is gradually integrated to get to location 1 and location 2.

As shown in FIG. 27, the electrode starts at location 3 and moves over to location 4. At location 3 the slope is positive. As the drive electrode is approached, the AC potential increases. As the electrode proceeds to the right, the value increases.

The slope reverses, decreasing until the electrode reaches location 4. There, the slope is flat. Eventually the slope starts increasing. The distance from location 3 to location 4 is computed simply by calculating the slope and the change in potential as the electrode position moves through the curve system.

The above explanation is demonstrative only. The actual calculations in a specific application are not necessarily as simple as the demonstrative example which shows the distance between two electrodes in two dimensions. In the body, these fields occupy three dimensions.

In order to more rigorously determine the electrodes' location, three different orthogonal fields are created. Fields which are not completely orthogonal but have some orthogonal nature can also be appropriate for this application. Each of these fields is provided in a different frequency. Employing a combination of slope and value in each of the frequencies allows calculation of the exact location of the electrodes.

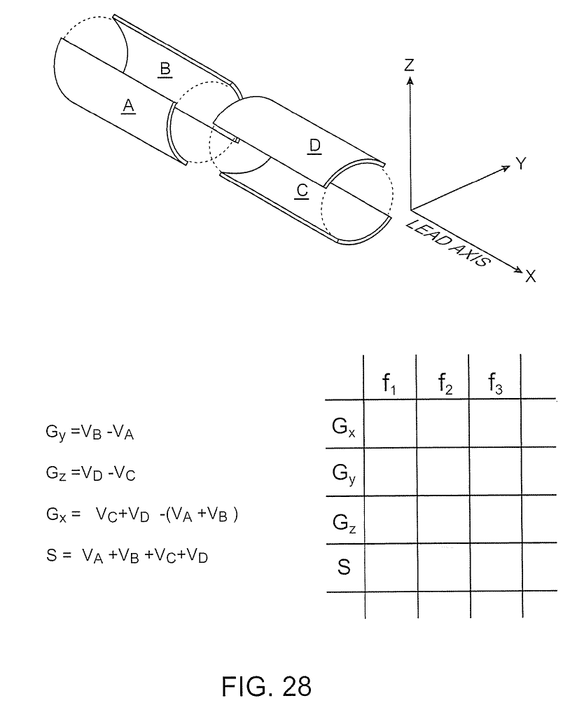

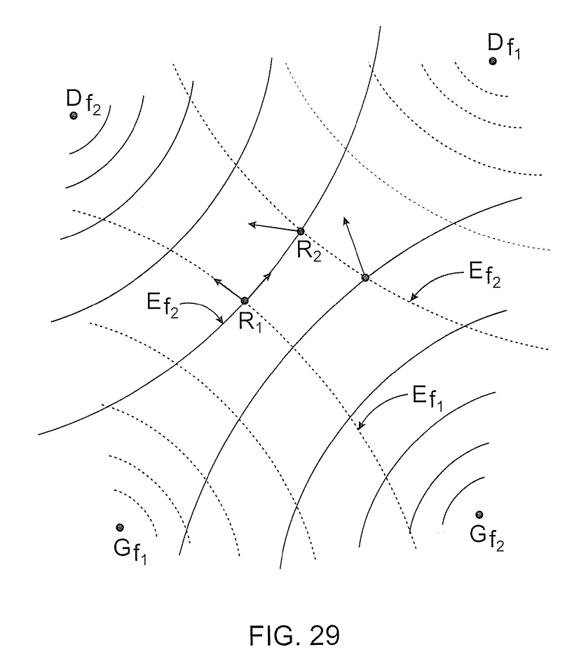

The design of one appropriate device for measuring the gradient and value of potential is shown in FIG. 28. Four electrodes are shown. Electrodes A and B are on opposite sides of the lead. Electrodes C and D are opposite from each other, but oriented 90 degrees apart from electrodes A and B.

Axis X is positioned down the length of the axis of the lead body housing the four electrodes. Axis Y, perpendicular to axis X, goes through electrodes A and B. Axis Z, perpendicular to both axis X and axis Y, runs though the centers of electrodes C and D. Additional electrode configurations of interest are disclosed in U.S. Patent Application Ser. No. 60/655,609 filed on Feb. 22, 2005; the disclosure of which is herein incorporated by reference.

To determine the gradient in axis Y, the AC voltage at electrode B is determined. AC voltage at electrode A is subtracted from the AC voltage at electrode B. The resulting absolute number is proportional to the gradient of the change in electrical potential and its changes over that dimension. In this case, that would be about 2 mm.

This analysis procedure is summarized as:

G_{y}=V_{B}−V_{A}

To determine the gradient in axis Z, the voltage at electrode D is determined. The voltage at electrode C is subtracted from that voltage. In both of these cases, the subtracting voltages is typically accomplished with a instrumentation amplifier. The amplifier takes the difference of the two voltages, and amplifies the difference by a factor, by example 1000. The signal is put into a lock-in amplifier. As a result, the noise from other signals is removed and only the value at the frequency of interest is recorded.

This analysis procedure is summarized as:

G_{z}=V_{D}−V_{C}

To determine the gradient along the lead axis, voltages at electrodes C and D are added. The sum of the voltages of electrode A and B are subtracted from this number. This calculation provides the gradient in the X direction, that is the difference going along axis X of the lead.

The value of the field at that frequency is determined by the sum of these voltages, that is voltage A plus voltage B plus voltage C plus voltage D. In practice, three different pairs of drive electrodes are located along different axis. Ideally, these electrode pairs would have three different orthogonal axis. One pair of these electrodes generates a gradient for each of those frequencies. This produces a gradient in the Y direction for frequency 1, a gradient in the Y direction for frequency 2, and a gradient in the Y direction for frequency 3. These values are all calculated simultaneously because lock-in amplifiers are employed for each of those three frequencies.

This analysis procedure is summarized as:

Gₓ=V_{C}+V_{D}−(V_{A}+V_{B})

FIG. 28 provides a table of gradient and frequency to better demonstrate these concepts, and provide one structure among many appropriate structures, for assessing the sum of the values. This approach is useful where three frequencies are broadcast from pairs of electrodes that are orthogonally placed relative to each other.

From these four electrodes, four values can be computed. These values are a gradient in the X direction, a gradient in the Y direction, a gradient in the Z direction, and the sum of all of them, which would be the value of that frequency at that location. This analysis procedure is summarized as:

S=V_{A}+V_{B}+V_{C}+V_{D}

FIG. 29 shows two pairs of drive electrodes operating at two different frequencies. The ground frequency G_{f1} is shown in the lower left hand corner, and drive frequency D_{f1} is shown in the upper right hand corner. The equal potential lines shown in solid lines. Drive frequency D_{f2} is in the upper left hand corner. Ground frequency G_{f2} is in lower right hand corner. The equal potential lines of that frequency are shown in dashed lines.

If the electrode is located conveniently at the intersection of two of these lines, the gradient at each of those frequencies can be measured. This gradient is provided as a vector of equal potential in each of these frequencies. The receive electrode at location R bears an arrow that is perpendicular to the equal potential lines of frequency f₁ and a black arrow which represents the vector pointing in towards the increasing potential of frequency f₂.

From the value and the gradient, the distance is determined. By example, the electrode is located at a position along equal potential line E_{f1}. The electrode is also on the equal potential line E_{f2} which are perpendicular to the electrode. From those two numbers, the electrode's location in space is determined.

As the electrode moves in space to another position, successive measurements are taken. The electrode moves to location R₁ from original location R₀. When the electrode is at location R₁ the gradient, that is the value of drive frequency f₂, has not changed. It is still on the same potential as drive frequency f₂. The gradient has changed direction slightly, and angle has changed so that it is still pointing towards drive frequency D_{f1}. The angle is slightly different, but otherwise it has not changed much.

On the other hand, with respect to drive frequency f₁, the electrode has moved from equal potential line E_{f1} to equal potential line E_{f2}. As that gradient is known, the distance from original location R₀ to location R₁ is calculated directly. This is accomplished by changes slope as it goes from original location R₀ to location R₁. This is similar to the one dimensional case described in the first set of figures. If the electrode then moves to location R₂, the gradient is in frequency f₂, the angle has changed again, and the value has changed significantly.

However, since the electrode has moved along the equal potential line E_{f2}, it has not changed potential in frequency f₁. From this it is computed that the electrode is going along the gradient of the second frequency. The distances of location R₁ and location R₂ are computed in a manner similar to that demonstrated in the one dimensional drawings discussed above. From these, a matrix of the gradients and values are computed. The locations of each of the electrodes is determined by methods similar to those described herein.

The different electrical gradient tomography embodiments of the present invention have common characteristics. There are two oppositely located pairs of electrodes whose positions are at 90° from each other. From those four electrodes, the electrical gradient in three dimensions, that is X, Y and Z, are computed. The absolute value of the electrodes is also computed at multiple frequencies, shown here as frequencies F1, F2, and F3.

From those 12 values of gradients, and values at three different frequencies, a signal change is developed that produces the location of that position within the body. As these values change, the motion from one location to another location is also measured.

FIG. 29 provides a simple example of this inventive embodiment in two dimensional space, where these teachings are readily adapted by those of skill in the art to three dimensional space.

### Magnetic Tomography

Aspects of the magnetic tomography embodiment of the present invention are similar to those of the electrical tomography discussed above. In representative magnetic tomography embodiments of the invention, once the magnetic field signals are converted to voltages, they are demodulated with a lock-in amplifier. At this point, the amplitude is a function of position. This commonality of data collection and processing among the various field embodiments of the present invention is made even more evident in the circuitry and data method section of the present application.

The difference between electrical and magnetic tomography is in how the fields are generated, how they are detected, and what the relevant fields are. For magnetic tomography the relevant field ψ is the magnetic vector field B. The magnetic field can be generated by a permanent magnet. However, in representative applications the magnetic field is easily and controllably generated by a multi-turned coil. The magnetic field may be detected using any convenient protocol, such as a coil, flux-gate, Hall-effect sensor, magneto-resistive device, or superconducting quantum interference device.

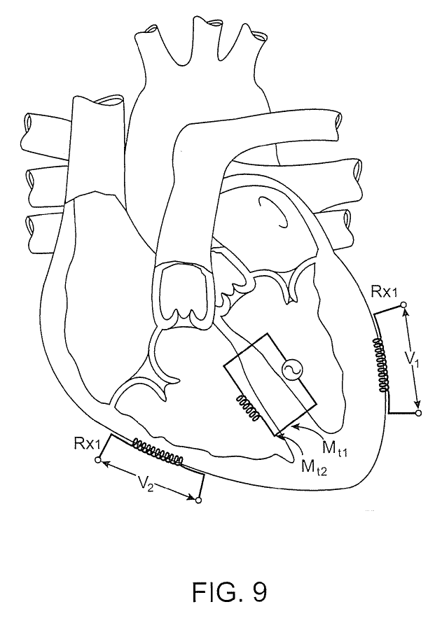

In the magnetic tomography embodiment of the present invention shown in FIG. 9, a magnetic coil acts as a dipole, serving as the source generator. Another magnetic coil is a dipole receiver, serving as the receive element. If an alternating current is passed through the coil, it will generate a magnetic field through the Faraday Law of Induction. This change in magnetic field induces electromotive forces in the received coil, which are detected.

One advantage of magnetic tomography over electrical tomography is that magnetic fields are not affected by the tissues nearly as much as the electric fields. The magnetic permittivity and permeability of the tissue is essentially unity for magnetic fields. Intervening tissues do not disturb magnetic fields at all, providing an essentially transparent medium for magnetic tomography.

The transparency of intervening tissue to a magnetic field allows for exact determination of distances. One can calculate the signal levels for various distances, and solve the inverse problem. Some of the present inventors have completed a calculation showing that the signals are about half a mille volt at 5 cm for a 100 turned coil. This size of a coil is comparable to those found in a 6 French catheter. Devices of this size are highly advantageous for use in the heart.

Despite a highly compact size, the voltage sensitivity is about 40 μvolts per millimeter. This is the change in voltage detected by the coil as it moves through the magnetic field generated by a different coil.

Tying back to the framework generalized in Table 1, a magnetic field \vec{B}(t,\vec{r}) is applied as the continuous field, which is described by the following formula:

\vec{B}(t,\vec{r})=A(\vec{r})sin(2πft+φ)

where amplitude is a function of position. In the case where the frequency is fixed, lock-in demodulation is used to determine the amplitude. Analogous to the electrical tomography embodiment, detection of phase shifts at higher frequencies can also be employed to glean tomography data.

One difference of the magnetic embodiment of the present invention over the use of electric field is that, whereas the voltage field is a scalar quantity, the magnetic is a vector quantity. As a result, to most effectively determine the vector orientation of the magnetic field, three coils are utilized, one for each dimension of real space. The three-coil approach allows determination the magnetic field vector.

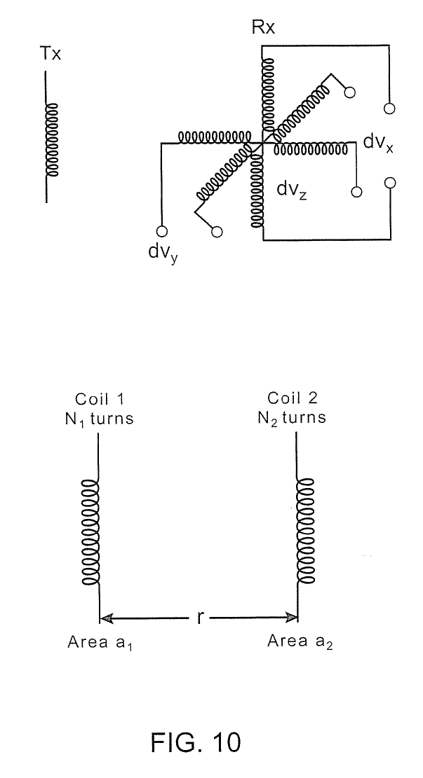

To address the full inverse problem, a three dimensional gradiometer is provided, as shown in FIG. 10. Given the known current through the transmit coil, a three dimensional gradiometer makes possible exact solution of position, both orientation and separation vector. Six (6) degrees of freedom are provided between the transmit coil and receive coil. In this manner, absolute distances are determined, such as between the septum of the heart and a free wall as a function of time. A reconstruction of an entire picture of wall position and movement is provided. This feature of the present invention is useful for determining cardiac synchrony and other critical cardiac parameters, as reviewed in greater detail below.

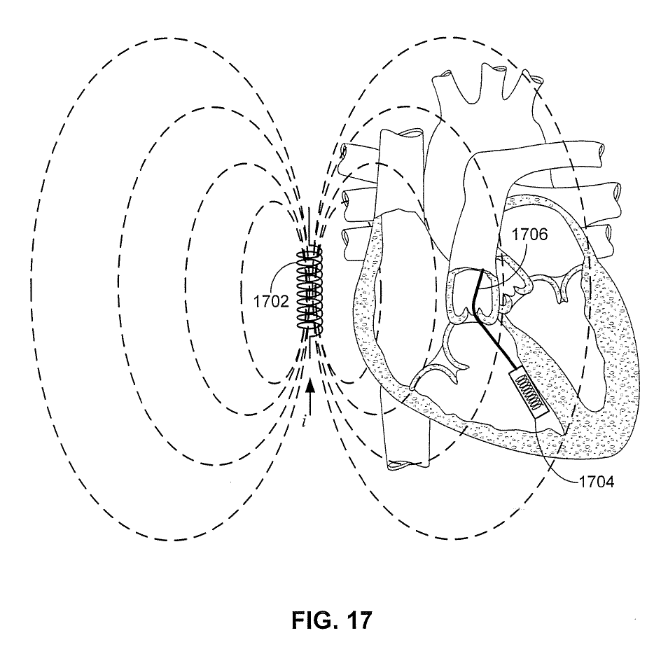

FIG. 17 illustrates an exemplary configuration for magnetic tomography using one inductor coil, in accordance with an embodiment of the present invention. A driving current i passes through a driving coil 1702, producing a magnetic field which encompasses the heart and the surrounding tissues. Correspondingly, magnetic field lines represented in dashed lines emanate from the north pole of driving coil 1702 and curve around to the south pole.

An electrode 1704 is located in the right ventricle of the heart and is coupled to a pacing lead 1706. Electrode 1704 also includes an inductor coil. The magnetic field induces a current in the inductor coil. Particularly, if i is a sinusoidal AC current, the magnetic field is also a rotating sinusoidal field with the same frequency. According to Faraday's law of induction, the induced current in the inductor coil is a sinusoidal AC current with the same frequency as well. Therefore, one can use a lock-in amplifier to detect the induction-current signal and subsequently can determine the location of electrode 1704 with reference to the existing magnetic field.

Because the intensity of a current induced in a coil is proportional to the magnetic flux captured by the coil, a single inductor coil may not be sufficient to indicate precisely an electrode's position. For example, in FIG. 17, the induced current may experience little change when electrode 1704 is near the waist of the magnetic field lines and is aligned in approximately the same direction. One embodiment of the present invention solves this problem by using a 3-D magnetic gradiometer.

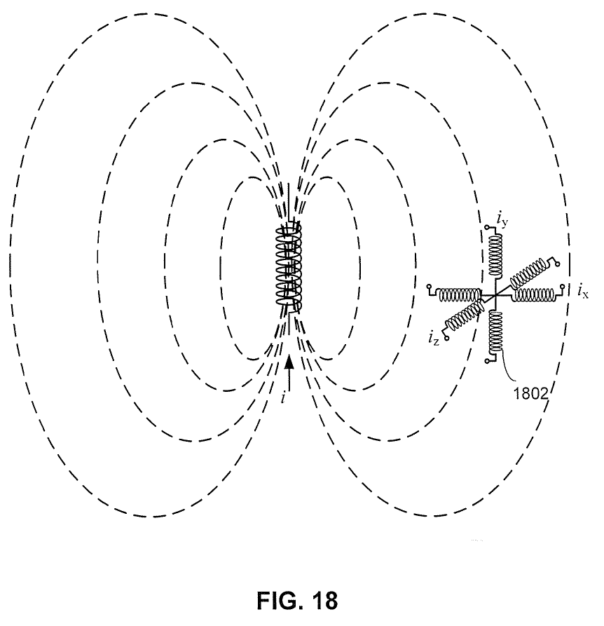

FIG. 18 illustrates an exemplary arrangement for 3-D magnetic tomography using a magnetic gradiometer, in accordance with an embodiment of the present invention. A 3-D magnetic gradiometer 1802 includes three pairs of opposite-facing inductor coils aligned in three substantially orthogonal directions. In each direction, the two opposite-facing coils are of opposite winding directions (e.g., one is wound clockwise and the other is wound counter-clockwise). When placed in a magnetic field, the two currents induced in the two coils flow in opposite directions. The net current in a pair of coils indicates the difference in the magnetic flux captured by the two coils. Instead of measuring the strength of the magnetic field, a pair of opposite-facing coils measure the changes in the magnetic field (i.e., the gradient of the magnetic flux) in one given direction. By using three orthogonal pairs of coils, one can measure the magnetic-field gradient in three directions, and can precisely locate an electrode containing the gradiometer.

### Electro-Magnetic Tomography

The above section provides a review of the manner in which amplitude and phase in electric and magnetic tomography can be determined by a lock-in amplifier. As noted, detection of amplitude can be readily employed at low frequencies of AC oscillation. In other embodiments, detection of phase is employed, e.g., at higher frequencies. At very high frequencies, e.g., above a few GHz, the corresponding wave length becomes shorter than typical dimensions of the body. This phenomenon provides an opportunity to observe a Doppler shift, not in the electric or magnetic fields individually, but in the electromagnetic field.

This electromagnetic field is detected with the same detection methods described above for the electric or the magnetic field. As there is essentially a wave propagating inside the body, there will be a Doppler shift associated with its velocity. In the unifying framework summarized in Table 1, there is an electromagnetic wave, either E(t) or B(t), which is a function of velocity. Whereas in the prior examples, the amplitude and phase differences were functions of position, in the case of electromagnetic tomography, there is a frequency that is a function of velocity.

FM demodulation is used to detect these small frequency differences with high precision. The actual sensing element can be selected from many different devices. For instance, the sensing element can be an electrode, an antenna that detects the electric field, or a coil that detects the magnetic field, among other possible detectors. Their signals are passed into the FM demodulator and the velocity as function of frequency is determined. There is a shift in velocity that is described by the following formula:

$${f}_{observed} + {f}_{generated} \sqrt{\frac{1 + \frac{V}{C}}{1 - \frac{V}{C}}} ,$$

where C is the speed of light. This velocity shift is fairly independent of the influence of intervening tissues. Because the exact frequency of the generating field is known, very fine measurements can be made when needed to exclude extraneous noise band width.

### Electrode Tomography System Operation

Since both electrical tomography and magnetic tomography involves detecting an induced sinusoidal signal on an electrode, the system operation for electrode tomography using either technology can be based upon similar principles. Therefore, although the examples herein are described with reference to an electrical tomography system, similar arrangements are readily apparent to those skilled in the art from the following description.

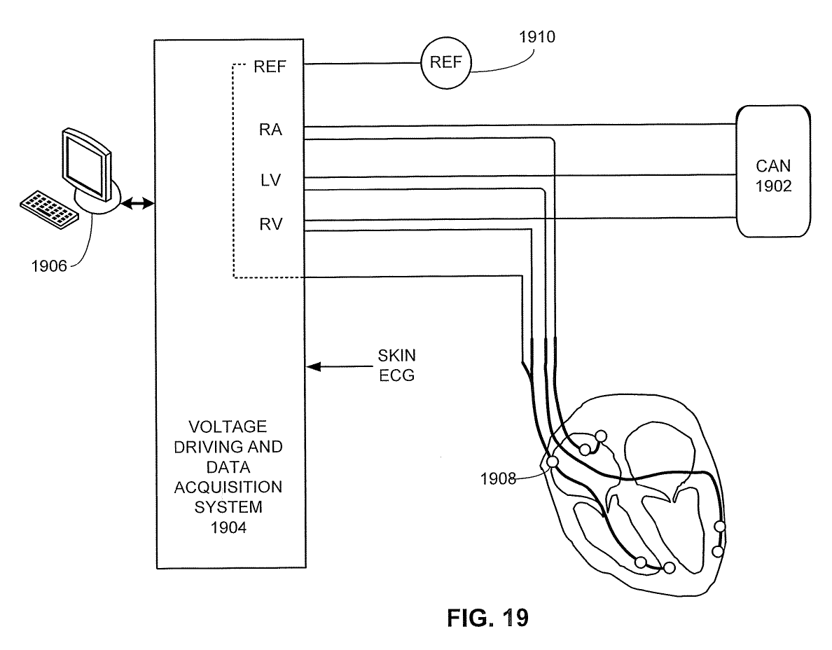

One advantage of an electrode tomography system applying an electrical field is that the system can operate on existing cardiac pacing system and, therefore, incurs minimum risk to a patient. FIG. 19 illustrates an electrical tomography system based on an existing pacing system, in accordance with an embodiment of the present invention. In this example, there are a number of pacing electrodes implanted in a patient's heart. These electrodes may be off-the-shelf electrodes for regular cardiac pacing purposes.

A voltage-driving and data-acquisition system 1904 couples to a pacing can 1902. System 1904 also couples to the electrodes which reside in the right atrium (RA), left ventricle (LV), and right ventricle (RV). Leads from pacing can 1902 are first routed to system 1904 and then routed to the electrodes. System 1904 can use the leads to drive any electrode, including pacing can 1902, and can detect induced signals on non-driving electrodes through the leads. System 1904 also has a reference port which may couple to an external voltage reference point, such as the ground. In the example in FIG. 19, electrode 1908 is coupled through the lead to the reference port, which is coupled to a ground reference voltage 1910.

The arrangement described above allows pacing can 1902 to send regular pacing signals to the electrode while performing electrical tomography. Such simultaneous operation is possible because pacing signals are typically short pulses, whereas the driving voltage is a constant sinusoidal signal with a well defined frequency. Furthermore, system 1904 may receive skin electrocardiogram (ECG) data to assist the analysis of the electrical tomography signals. System 1904 also interfaces with a computer 1906, which performs analysis based on the collected data.

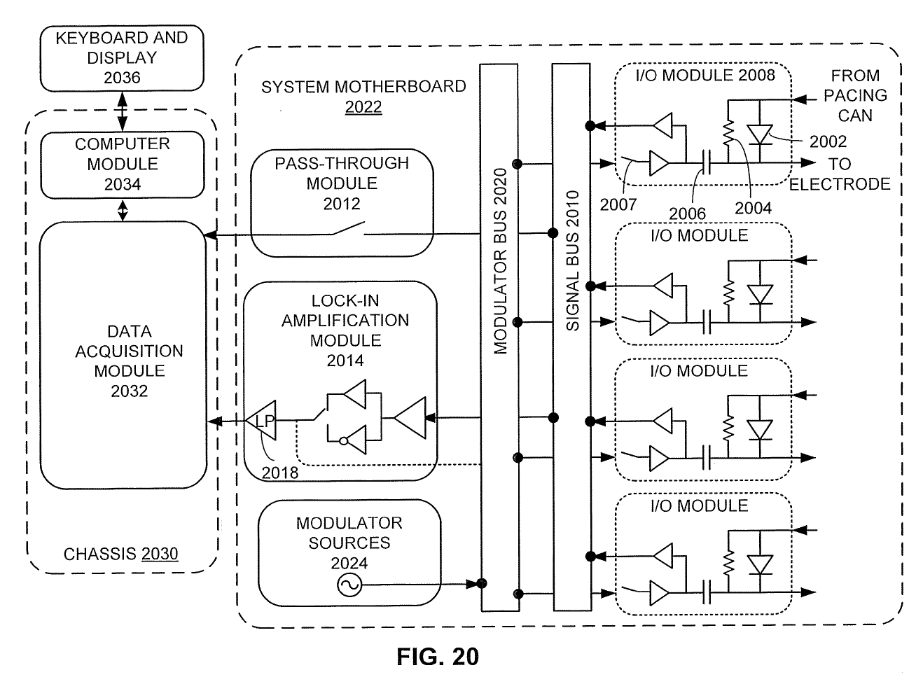

FIG. 20 illustrates a schematic circuit diagram for the voltage-driving and data-acquisition system 1904 in FIG. 19, in accordance with an embodiment of the present invention. The system includes a system motherboard 2022 and a chassis 2030. System motherboard 2022 accommodates a number of input/output (I/O) modules, such as I/O module 2008. Also included on system motherboard 2022 are a signal bus 2010, a modulator bus 2020, a pass-through module 2012, a lock-in amplification module 2014, and a set of modulator sources 2024.

An I/O module may contain a number of I/O circuits, each serving one data channel. The I/0 circuit in I/O module 2008 has a loop-back stage which includes a diode 2002 and a resistor 2004. Resistor 2004 and diode 2002 allow a pacing signal from the pacing can to pass through and reach the electrode. In addition, resistor 2005 and diode 2002 serves to isolate the AC driving voltage used by the tomography system from the pacing can.

A coupling capacitor 2006 allows receipt of induced AC signals from an electrode. Capacitor 2006 also couples a driving AC voltage to an electrode when the electrode serves as a driving electrode. Correspondingly, switch 2007 is engaged when the coupled electrode is a driving electrode, and is disengaged when the coupled electrode is a sensing electrode.

When receiving signals, I/O module 2008 transmits the received AC signals to the signal bus 2010, which subsequently transmits the received signals to lock-in amplification module 2014. When used for driving an AC voltage, I/O module 2008 receives an AC voltage from the modulator bus 2020. Note that modulator sources 2024 include a number AC voltage sources and can drive multiple electrodes simultaneously. Accordingly, modulator bus 2020 is responsible for routing the AC driving voltages to proper I/O modules.

Lock-in amplification module 2014 includes multiple lock-in amplifier circuits. In a lock-in amplifier circuit, an input signal is first amplified, and then multiplied by a signal with a reference frequency to produce a product signal. When the input signal is a detected AC signal induced on an electrode, the corresponding AC driving voltage is used as the reference signal, so that the product signal has a DC component that reflects the level of the induced AC signal. The product signal is then filtered by a low-pass filter 2018 to remove any noise at other frequencies, including a pacing pulse. Furthermore, pass-through module 2012 transmits the received signals directly to data acquisition module 2032 without any lock-in amplification.

Chassis 2030 includes the data acquisition module 2032 and a computer module 2034. Data acquisition module 2032 digitizes the received signals and transfers the data to computer module 2034. Computer module 2034 may include a central processing unit (CPU), a memory, and a hard drive, and is responsible for storing and analyzing the data. A keyboard and a display 2036 interfaces with computer module 2034 to facilitate data input and output.

### Common Mode Rejection

One challenge in detecting small signals induced upon an electrode is the common mode problem. Particularly, when two electrodes submerged in blood (or surrounded by organic tissue) are used to drive an AC voltage, the impedance-between the two electrodes is dominated by the impedance at the interface between the electrode and the blood (or organic tissue). For example, the impedance between an electrode and blood can be on the order of several kilo Ohms, whereas the impedance of the blood is only on the order of several hundred Ohms. This dominating interface impedance results in a large voltage drop at the interface. Any variation of this interface impedance can cause the field strength across the tissue region to vary significantly. The resulting voltage variation can easily overwhelm any change in the signal induced upon the target electrode whose location is to be determined.

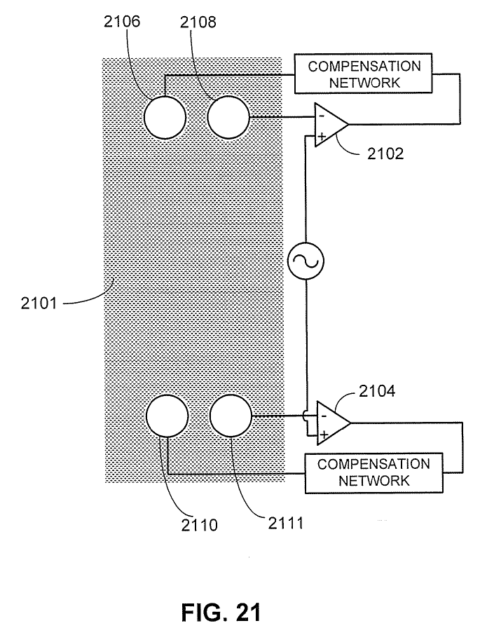

FIG. 21 illustrates one embodiment of the present invention that eliminates the effect of large electrode interface impedance by using four electrodes for driving an AC voltage. Two driving electrodes, 2106 and 2110, are submerged in blood (or organic tissue) 2101. Two auxiliary electrodes, 2108 and 2111, are placed in the vicinity of electrodes 2106 and 2110, respectively.

To eliminate the effect of large interface impedance of electrodes 2106 and 2110, and to obtain a stable AC voltage drop across the blood (or tissue) 2101, the system facilitates two operational amplifiers (OPAMPs) 2102 and 2104. The positive input of OPAMP 2102 is coupled to auxiliary electrode 2108, and the positive input of OPAMP 2104 is coupled to auxiliary electrode 2111. An AC voltage source is coupled between the two negative inputs of the two OPAMPs. Driving electrode 2106 is coupled to the output of OPAMP 2102. Correspondingly, driving electrode 2110 is coupled to the output of OPAMP 2104.

With this configuration, there remains a stable AC voltage drop between auxiliary electrodes 2108 and 2111, because the two inputs of an OPAMP have substantially the same electric potential. Moreover, although there is also a large interface impedance around auxiliary electrodes 2108 and 2111, there is only negligible current flowing through the two positive OPAMP inputs. Therefore, the voltage drop due to large interface impedance of auxiliary electrodes 2108 and 2111 is minimal. Consequently, the voltage drop across blood (or tissue region) 2101 remains the same as the driving AC voltage.

The voltage difference between driving electrodes 2106 and 2110, however, may not be a constant value. This is because the current flowing through the blood is kept constant (because the voltage drop between auxiliary electrodes 2108 and 2111 is constant, and because the blood impedance typically remains stable), whenever there is variation in the interface impedance of driving electrode 2106 or 2110, the voltages on these driving electrode also change correspondingly. Nevertheless, the total voltage drop across the blood region is stable, which facilitates detection of changes in an induced voltage of a target electrode whose location is to be determined.

Other types of common-mode interference may also be present. For example, the driving electrodes and auxiliary electrodes may move with the tissue and thus change the voltage distribution. One way to mitigate this common-mode effect is to measure the difference of the induced signals on several target electrodes, instead of the absolute value of the induced signal on a single target electrodes. This comparative method, however, may require careful calibration of the gain of each lock-in amplifier for each target electrode.

### Simultaneous Transmission of Multiple Tomography Signals Over One Wire

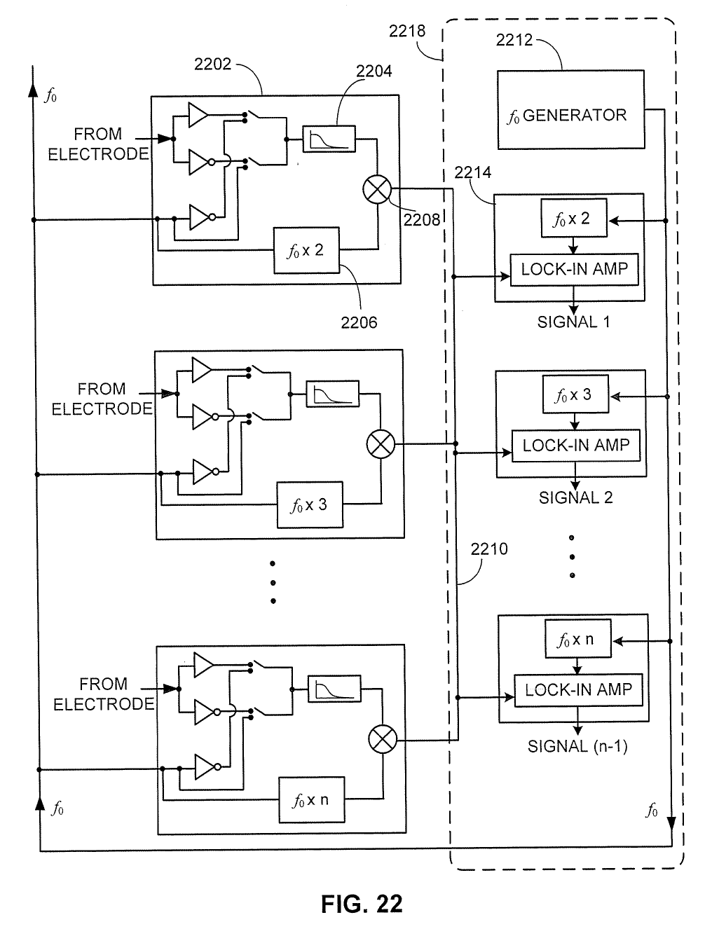

FIG. 22 illustrates one embodiment of the present invention that enables simultaneous transmission of tomography signals over a single wire using frequency division multiplexing. During operation, the system applies an AC voltage with a base frequency f₀ across the tissue region. Every electrode is equipped with a multiplexer module, such as module 2202. A module has two inputs: one from the electrode for the tomography signal, and one for the base frequency f₀.

For example, in module 2202, the tomography signal is first amplified and then multiplied with the base frequency f₀. Note that in the example shown in FIG. 22, module 2202 also facilitates two switches, which enable an arbitrary selection of the sign for the tomography signal and the base-frequency signal. A low-pass filter 2204 then filters the multiplied signal. The cut-off frequency of low-pass filter 2204 is approximately the same as the base frequency f₀ (e.g., 100 KHz). Therefore, low-pass filter 2204 can use a capacitor with a more compact size, which allows module 2202 to reside

Meanwhile, a frequency multiplier 2206 multiplies the base frequency and produces a carrier frequency 2f₀, which is specific to module 2202. A frequency mixer 2208 subsequently mixes the filtered signal with the carrier frequency, and transmits the output signal to a common signal-return wire 2210.

Within each frequency-division-multiplexer module, the frequency multiplier multiplies the base frequency with a different factor. Consequently, the tomography signal from every electrode is carried by a different carrier frequency, i.e., 2f₀, 3f₀, . . . , nf₀. The system can therefore simultaneously transmit multiple tomography signals over a signal wire with minimum cross talk between the signals.

The demultiplexer circuits may reside in an external system 2218 or in a pacing can. For each tomography signal, there is a demultiplexer module, such as demultiplexer module 2214. Within a demultiplexer module is a frequency multiplier that produces a carrier frequency same as the carrier frequency for a tomography signal, using the same base frequency f₀. Also included in a demultiplexer module is a conventional lock-in amplifier operating at the carrier frequency supplied by the frequency multiplier. In this way, the system can demultiplex the mixed signals at different carrier frequencies and reproduce each tomography signal. In addition, demultiplexing system 2218 may also include a base-frequency generator 2212 that provides the f₀ signal to the demultiplexer modules as well as the multiplexer modules.

### Pressure Field Tomography

Sound is a pressure field. Using pressure as the continuous field in the present tomography invention, the pressure field is a function of time. All three detection methods set forth in Table 1, i.e., amplitude, phase and frequency, can be used to measure sound.

As with the above reviewed continuous field embodiments, sound generates a continuous field as described by:

ψ=A sin(2πft+φ)

Either A, f, or φ is a function of an interesting parameter

P(t,v)=A sin(2πf(v)+φ). (in representative embodiments where the change in f, is small, FM demodulation is employed

In the case of pressure field tomography, a transducer is selected depending on engineering and application parameters. By example, for ultrasound, a piezoelectric crystal which generates a pressure wave in the tissues of the body would be appropriate. Alternately, miniature acoustic transducers and other sound producers can be employed.

In representative embodiments, the pressure wave is detected by another piezoelectric transducer. In a simple embodiment, the frequency shift is observed. In one example, two leads are provided, each with one of these piezoelectric transducers on them moving relative to one another. As a result, there will be a Doppler shift in the frequency. It will be provided as:

$${f}_{observed} + {f}_{generated} \frac{1}{1 + \frac{V}{C}} ,$$

where C is the velocity of sound in the medium.

This frequency can be demodulated and the velocity determined.

The amplitude and phase of the pressure field can also be utilized to glean tomography data. There is an attenuation factor to the sound as it travels through the tissues. There is also a factor that comes from the sound spreading though the tissues. By understanding these, it can be determined the amplitude would change as a function of position.

Additionally, the phase changes as a function of velocity. A lock-in detection or some interferometer technique is employed to determine the phase change.

### Light Tomography

Light, along with the frequency applications of electrical and magnetic tomography, is classified as an electromagnetic wave. However, the characteristics of light provide special applications and opportunities in the present invention due to light's inherent, often unique, characteristics.

The many diverse techniques available for dealing with light allow detection of extremely faint signals and provide precise determinations of the characteristics of the signals. These techniques are well known to the ordinary skilled artisan

In representative light tomography embodiments, a light field generation element (i.e., a light emitter), such as an LED or a laser, is provided at a first location, e.g., on one lead. A light receptor, such as a photo diode, is provided at the tissue location of interest, e.g., on another lead stably associated with the target tissue location of interest. The change in amplitude as it is attenuated by the tissue provides the necessary data.

With a light source on one lead and a light receiver on the other, there are two effects which dictate the intensity of the received light. The first is a simple spreading of the light as it falls from a point source. The other effect is an attenuation as intervening tissues absorb and scatter light.

The spreading of the light as it falls from a point source goes as 1/r2 and would exist for either an LED or a un-collimated laser. This effect would not occur for a collimated laser. The other effect dictating the intensity of the received light is an attenuation effect due to intervening tissue absorption and light scattering. This attenuation factor is exponential. There will be some attenuation to be considered at any wavelength. There are particular wavelengths of light in the near infrared where light travels relatively unimpeded with little attenuation through the body tissues. Thus, the light intensity reducing effect is relatively small in the near infrared range. Accordingly, near infrared ranges can be selected to mitigate the attenuation effect. Such wavelengths provide a desirable window for light tomography. Nonetheless, this effect is still present with a scattering depth of several centimeters. In representative embodiments where light of a near infrared range is employed, the light has wavelength ranging from about 500 to about 2000 nm.

In order to ascertain distance, interaction of these two effects are calibrated or calculated. The tomography system is then designed to glean clear tomography information, such as by adjusting the raw data to account for the effects, and provide useful information, or otherwise engineering the system to both compensate for and exploit these effects.

In a region where the space in between the receiver and the source is less than scattering length, the 1/r2 factor is dominant. In a region where the receiver and the source are several scattering lengths away, the exponential factor would be dominant. In the middle, both factors are considered to optimize the efficacy of the tomography device and data.

By quantifying attenuation, position as a function of the received light level is determined. Additionally, modulation of the light allows a lock-in detection in addition to other features in order to filter out extraneous signals.

There is a phase shift as the two leads move relative to each other. This is detected through interferometer methods. Interferometer methods are well established for determining the phase shifting in a beam of light, and are well known to the ordinary skilled artisan.

As the source moves relative to the receiver, there will be a frequency shift. This phenomenon was discussed in the electromagnetic wave case, above. However, in the range of light, much higher frequencies are encountered. Terahertz up to hundreds of terahertz are present. Despite these extremely high frequencies, however, for frequency shift in the near field, the wavelength is much shorter than the separation between the electrodes. Thus, a frequency shift is observed in the light spectrum electromagnetic wave. Homodyne detection is used to measure that frequency shift very precisely in an interferometer method. This approach extracts extremely fine frequency shifts, providing fine measure of the relative velocity of the two sources.

### Thermo Field Tomography

In the case of thermo field tomography, two sources are provided; a heat source and a reference. These sources can be of a range of devices, such as Peltier-coolers, thermo electric coolers, and the like. A temperature gradient is generated between the generator and the reference. By forcing the sources to be slightly different in temperature, a thermal gradient is generated. In representative embodiments, the thermal gradient has a magnitude ranging from about 0.1 to about 2° C./cm, e.g., about 1° C./cm. A very sensitive temperature sensor is introduced which measures where along that gradient it is positioned.

Where amplitude is the parameter of interest, the amplitude of the temperature varies as a function of position. By analogy to the embodiments discussed previously, this temperature gradient is modulated in an “AC” fashion. Amplitude is most easily detected in thermo field tomography. Where phase is the parameter of interest, phases are detected as a function of velocity.

### Additional Features Found in Representative Systems.

Embodiments of the subjects systems incorporate other physiologic sensors in order to improve the clinical utility of wall-motion data provided by the present invention. For example, an integrated pressure sensor could provide a self-optimizing cardiac resynchronization pacing system with an important verification means, since wall motion optimization in the face of declining systemic pressure would be an indication of improper pacing, component failure or other underlying physiologically deleterious condition (e.g., hemorrhagic shock). One or more pressure sensors could also provide important information used in the diagnosis of malignant arrhythmias requiring electrical intervention (e.g., ventricular fibrillation). Incorporation of other sensors is also envisioned.

In certain embodiments, the systems may include additional elements and features, such as a multiplexed system of the assignee corporation of the present application. This multiplexed system is described in part in currently pending patent applications U.S. patent application Ser. No. 10/764,429 entitled “Method and Apparatus for Enhancing Cardiac Pacing”, U.S. patent application Ser. No. 10/764,127 entitled “Methods and Systems for Measuring Cardiac Parameters”, and U.S. patent application Ser. No. 10/764,125 entitled “Method and System for Remote Hemodynamic Monitoring”, all filed Jan. 23, 2004, U.S. patent application Ser. No. 10/734,490 entitled “Method and System for Monitoring and Treating Hemodynamic Parameters” filed Dec. 11, 2003, U.S. Provisional Patent Application 60/638,692 entitled “High Fatigue Life Semiconductor Electrodes” filed Dec. 22, 2004, and U.S. Provisional Patent Application 60/638,928 entitled “Methods and Systems for Programming and Controlling a Cardiac Pacing Device” filed Dec. 23, 2004. These applications are herein incorporated into the present application by reference in their entirety.

Some of the present inventors have developed Doppler, pressure sensors, additional wall motion, and other cardiac parameter sensing devices. Some of these are embodied in currently filed provisional applications; “One Wire Medical Monitoring and Treating Devices”, U.S. Provisional Patent Application No. 60/607280 filed Sep. 2, 2004, U.S. patent applications Ser. No. 11/025,876 titled “Pressure Sensors having Stable Gauge Transducers”; U.S. patent application Ser. No. 11/025,366 “Pressure Sensor Circuits”; U.S. patent application Ser. No. 11/025,879 titled “Pressure Sensors Having Transducers Positioned to Provide for Low Drift”; U.S. patent application Ser. No. 11/025,795 titled “Pressure Sensors Having Neutral Plane Positioned Transducers”; U.S. patent application Ser. No. 11/025,657 titled “Implantable Pressure Sensors”; U.S. patent application Ser. No. 11/025,793 titled “Pressure Sensors Having Spacer Mounted Transducers”; “Stable Micromachined Sensors” U.S. Provisional Patent Application 60/615117 filed Sep. 30, 2004, “Amplified Complaint Force Pressure Sensors” U.S. Provisional Patent Application No. 60/616706 filed Oct. 6, 2004, “Cardiac Motion Characterization by Strain Measurement” U.S. Provisional Patent Application filed Dec. 20, 2004, and PCT Patent Application entitled “Implantable Pressure Sensors” filed Dec. 10, 2004, “Shaped Computer Chips with Electrodes for Medical Devices” U.S. Provisional Patent Application filed Feb. 22, 2005, Fiberoptic Cardiac Wall Motion Timer U.S. Provisional Patent Application 60/658445 filed Mar. 3, 2005, “Shaped Computer Chips with Electrodes for Medical Devices” U.S. Provisional Patent Application filed Mar. 3, 2005, U.S. Provisional Patent Application entitled “Cardiac Motion Detection Using Fiberoptic Strain Gauges” filed Mar. 31, 2005. These applications are incorporated in their entirety by reference herein.

Some of the present inventors have developed a variety of display and software tools to coordinate multiple sources of sensor information. Examples of these can be seen in U.S. Provisional Patent Applications “Automated Using Electromechanical Delay”, both filed Mar. 31, 2005. These applications are incorporated in their entirety by reference herein.

The present invention permits use of intracorporeal electrodes for the added purposes described even if these electrodes are primarily intended for other applications (e.g., cardiac pacing). Some of the embodiments described employ permanently implanted devices, while others employ acute use. Cardiac wall motion is detected by fixing catheters in relation to the cardiac wall of interest. However, localization of the catheters themselves is an intrinsic attribute of the system. Therefore, catheter localization can also be accomplished. For example, one or more temporary electrophysiology catheter electrodes could be employed for additional sensing using a permanently implantable embodiment of the system for generating electrical field(s). Using the extracorporeal display system to communicate with the implantable component and incorporating the temporary sense electrodes, the system could provide non-fluoroscopic catheter localization. Additionally, if the temporary catheter were temporarily fixed in association with an otherwise unmonitored cardiac wall location, additional cardiac wall motion data would be generated in the course of an invasive cardiac study

In the implantable embodiments of this invention, as desired wall motion, pressure and other physiologic data can be recorded by an implantable computer. Such data can be periodically uploaded to computer systems and computer networks, including the Internet, for automated or manual analysis.

Uplink and downlink telemetry capabilities may be provided in a given implantable system to enable communication with either a remotely located external medical device or a more proximal medical device on the patient's body or another multi-chamber monitor/therapy delivery system in the patient's body. The, stored physiologic data of the types described above as well as real-time generated physiologic data and non-physiologic data can be transmitted by uplink RF telemetry from the system to the external programmer or other remote medical device in response to a downlink telemetry transmitted interrogation command. The real-time physiologic data typically includes real time sampled signal levels, e.g., intracardiac electrocardiogram amplitude values, and sensor output signals including dimension signals developed in accordance with the invention. The non-physiologic patient data includes currently programmed device operating modes and parameter values, battery condition, device ID, patient ID, implantation dates, device programming history, real time event markers, and the like. In the context of implantable pacemakers and ICDs, such patient data includes programmed sense amplifier sensitivity, pacing or cardioversion pulse amplitude, energy, and pulse width, pacing or cardioversion lead impedance, and accumulated statistics related to device performance, e.g., data related to detected arrhythmia episodes and applied therapies. The multi-chamber monitor/therapy delivery system thus develops a variety of such real-time or stored, physiologic or non-physiologic, data, and such developed data is collectively referred to herein as “patient data”.

### Utility

The continuous field tomography methods of evaluating tissue location movement find use in a variety of different applications. As indicated above, an important application of the subject invention is for use in cardiac resynchronization, or CRT, also termed biventricular pacing. As is known in the art, CRT remedies the delayed left ventricular mechanics of heart failure patients. In a desynchronized heart, the interventricular septum will often contract ahead of portions of the free wall of the left ventricle. In such a situation, where the time course of ventricular contraction is prolonged, the aggregate amount of work performed by the left ventricle against the intraventricular pressure is substantial. However, the actual work delivered on the body in the form of stroke volume and effective cardiac output is lower than would otherwise be expected. Using the subject continuous field tomography approach, the electromechanical delay of the left lateral ventricle can be evaluated and the resultant data employed in CRT, e.g., using the approaches reviewed above and/or known in the art and reviewed at Col. 22, lines 5 to Col. 24, lines 34 of U.S. Pat. No. 6,795,732, the disclosure of which is herein incorporated by reference.

In a fully implantable system the location of the pacing electrodes on multi electrode leads and pacing timing parameters are continuously optimized by the pacemaker. The pacemaker frequently determines the location and parameters which minimizes intraventricular dyssynchrony, interventricular dyssynchrony, or electromechanical delay of the left ventricle lateral wall in order to optimize CRT. This cardiac wall motion sensing system can also be used during the placement procedure of the cardiac leads in order to optimize CRT. An external controller could be connected to the cardiac leads and a skin patch electrode during placement of the leads. The skin patch acts as the reference electrode until the pacemaker is connected to the leads. In this scenario, for example, the optimal left ventricle cardiac vein location for CRT is determined by acutely measuring intraventricular dyssynchrony.

The subject methods and devices can be used to adjust a resynchronization pacemaker either acutely in an open loop fashion or on a nearly continuous basis in a closed loop fashion.

Other uses for this system are as an ischemia detector. It is well understood that in the event of acute ischemic events one of the first indications of such ischemia is akinesis, i.e., decreased wall motion of the ischemic tissue as the muscle becomes stiffened. A Wall motion system would be a very sensitive indicator of an ischemic process, by ratio metrically comparing the local wall motion to a global parameter such as pressure; this has been previously described in another Proteus patent. One can derive important information about unmonitored wall segments and their potential ischemia. For example, if an unmonitored section became ischemic, the monitored segment would have to work harder and have relatively greater motion in order to maintain systemic. pressure and therefore ratio metric analysis would reveal that fact.

Another application of such position indicators that record wall motion is as a superior arrhythmia detection circuit. Current arrhythmia detection circuits rely on electrical activity within the heart. Such algorithms are therefore susceptible to confusing electrical noise for an arrhythmia. There is also the potential for misidentifying or mischaracterizing arrhythmia based on electrical events when mechanical analysis would reveal a different underlying physiologic process. Therefore the current invention could also be adapted to develop a superior arrhythmia detection and categorization algorithm.

Additional applications in which the subject invention finds use include, but are not limited to: the detection of electromechanical dissociation during pacing or arrhythmias, differentiation of hemodynamically significant and insignificant ventricular tachycardias, monitoring of cardiac output, mechanical confirmation of capture or loss of capture for autocapture algorithms, optimization of multi-site pacing for heart failure, rate responsive pacing based on myocardial contractility, detection of syncope, detection or classification of atrial and ventricular tachyarrhythmias, automatic adjustment of sense amplifier sensitivity based on detection of mechanical events, determination of pacemaker mode switching, determining the need for fast and aggressive versus slower and less aggressive anti-tachyarrhythmia therapies, or determining the need to compensate for a weakly beating heart after therapy delivery (where these representative applications are reviewed in greater detail in U.S. Pat. No. 6,795,732, the disclosure of which is herein incorporated by reference), and the like.

In certain embodiments, the subject invention is employed to overcome barriers to advances in the pharmacologic management of CHF, which advances are slowed by the inability to physiologically stratify patients and individually evaluate response to variations in therapy. It is widely accepted that optimal medical therapy for CHF involves the simultaneous administration of several pharmacologic agents. Progress in adding new agents or adjusting the relative doses of existing agents is slowed by the need to rely solely on time-consuming and expensive long-term, morbidity and mortality trials. In addition, the presumed homogeneity of clinical trial patient populations may often be erroneous since patients in similar symptomatic categories are often assumed to be physiologically similar. It is desirable to provide implantable systems designed to capture important cardiac performance and patient compliance data so that acute effects of medication regimen variation may be accurately quantified. This may lead to surrogate endpoints valuable in designing improved drug treatment regimens for eventual testing in longer-term randomized morbidity and mortality studies. In addition, quantitative hemodynamic analysis may permit better segregation of drug responders from non-responders thereby allowing therapies with promising effects to be detected, appropriately evaluated and eventually approved for marketing. The present invention allows for the above. In certain embodiments, the present invention is used in conjunction with the Pharma-informatics system, as described in U.S. Provisional Application Ser. No. 60/676,145 filed on Apr. 28, 2005 and U.S. Provisional Application Ser. No. 60/694,078; the disclosures of which are herein incorporated by reference.

Non-cardiac applications will be readily apparent to the skilled artisan, such as, by example, measuring the congestion in the lungs, determining how much fluid is in the brain, assessing distention of the urinary bladder. Other applications also include assessing variable characteristics of many organs of the body such as the stomach. In that case, after someone has taken a meal, the present invention allows measurement of the stomach to determine that this has occurred. Because of the inherently numeric nature of the data from the present invention, these patients can be automatically stimulated to stop eating, in the case of overeating, or encouraged to eat, in the case of anorexia. The present inventive system can also be employed to measure the fluid fill of a patient's legs to assess edema, or other various clinical applications.

### Computer Readable Medium

One or more aspects of the subject invention may be in the form of computer readable media having programming stored thereon for implementing the subject methods. The computer readable media may be, for example, in the form of a computer disk or CD, a floppy disc, a magnetic “hard card”, a server, or any other computer readable media capable of containing data or the like, stored electronically, magnetically, optically or by other means. Accordingly, stored programming embodying steps for carrying-out the subject methods may be transferred or communicated to a processor, e.g., by using a computer network, server, or other interface connection, e.g., the Internet, or other relay means.

More specifically, computer readable medium may include stored programming embodying an algorithm for carrying out the subject methods. Accordingly, such a stored algorithm is configured to, or is otherwise capable of, practicing the subject methods, e.g., by operating an implantable medical device to perform the subject methods. The subject algorithm and associated processor may also be capable of implementing the appropriate adjustment(s).

Of particular interest in certain embodiments are systems loaded with such computer readable mediums such that the systems are configured to practice the subject methods.

### Kits

As summarized above, also provided are kits for use in practicing the subject methods. The kits at least include a computer readable medium, as described above. The computer readable medium may be a component of other devices or systems, or components thereof, in the kit, such as an adaptor module, a pacemaker, etc. The kits and systems may also include a number of optional components that find use with the subject energy sources, including but not limited to, implantation devices, etc.

In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more, components are present in the same or different containers, as may be convenient or desirable.

The following examples are offered by way of illustration and not by way of limitation.

### EXPERIMENTAL

### I. Representative Animal Study

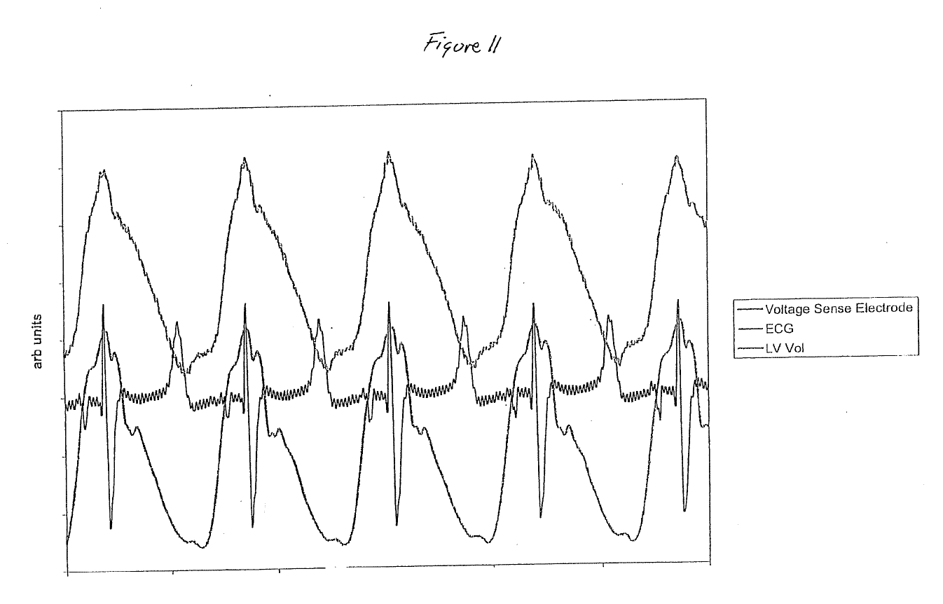

FIG. 11 provides a plot of data taken in a pig using the technique according to the invention. The trace marked “Voltage Sense Electrode” is the measurement taken using a lead in the RV apex as the driving electrode, a lead in the cardiac vein on the LV freewall as the receiving electrode, and a subcutaneous metal plate as the reference electrode. The receiving electrode signal was fed into a lock-in amplifier (a Stanford Research Systems model SR830). For comparison, the ECG and the LV volume (measured with a commercial pressure-volume catheter) are shown. It can be seen that the voltage sense signal is highly correlated to the LV volume (R=0.98).

### II. Principle Component Analysis of Cardiac Motion

Because various embodiments of electrode tomography as described herein facilitate simultaneous measurement of locations of multiple electrodes, advanced analysis of the tomography data is now possible. One embodiment of the presentation provides a method for analyzing basic modes of cardiac motion using principle component analysis. An experiment applying the principle component analysis is described below.

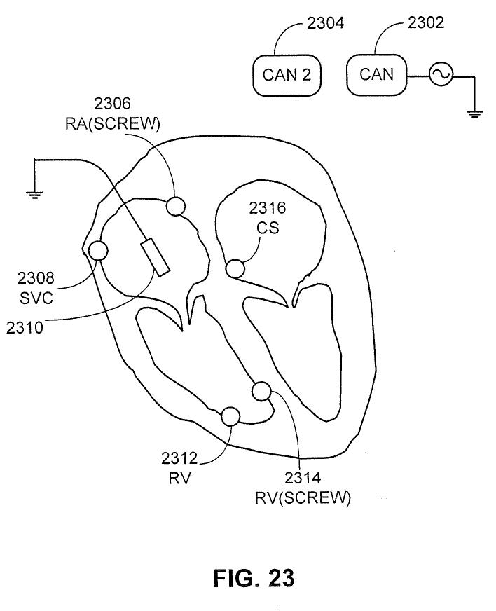

FIG. 23 illustrates the locations of electrodes used in an experiment performed in a pig heart demonstrating the analysis of electrical tomography signals according to one embodiment. The system drives an AC voltage between a can 2302 and a defibrillator coil 2310. The sensing targets are: an electrode placed on the superior vena cava (SVC) 2308, an electrode screwed into the right atrium (RA(SCREW)) 2306, an electrode screwed into the right ventricle (RV(SCREW)) 2314, an electrode placed near the coronary sinus (CS) 2316, an electrode placed in the right ventricle (RV) 2312, and a clip 2304 on the skin next to the can 2302 that acts like a second can (CAN2) (note that CAN2 is considered as one of the electrodes here).

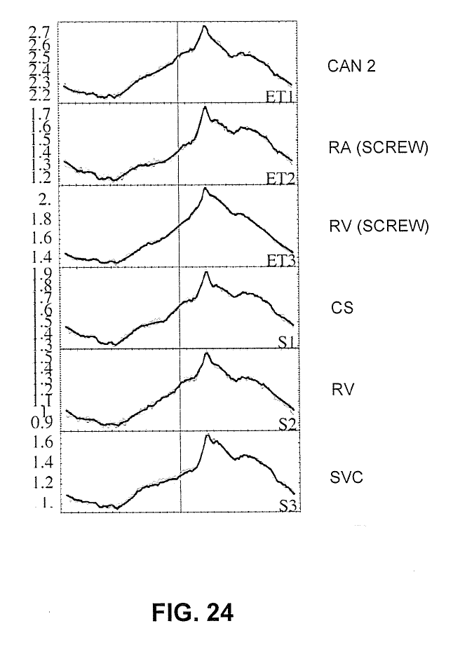

FIG. 24 presents the time-series plots for measured voltages of six target electrodes in the experiment as shown in FIG. 23. The plots are substantially similar, suggesting a strong common mode among all the electrodes. Next, a 6×6 correlation matrix is formed based on these six time series. An element Xᵢⱼ of the correlation matrix is defined as:

$${χ}_{ij} = \frac{1}{{t}_{2} - {t}_{1}} \cdot {\int }_{{t}_{1}}^{{t}_{2}} {s}_{i} ( t ) \cdot {s}_{j} ( t ) d t$$

where t₁ and t₂ denote the start and the end of the given time period, and sᵢ(t) denotes the time series of induced voltage on electrode i. (CAN2, RA(SCREW), RV(SCREW), CS, RV, and SVC are each assigned index 1, 2, 3, 4, 5, and 6, respectively.)

One can subsequently solve for the eigenvectors and eigenvalues of the correlation matrix. TABLE 2 presents the solution, sorted in a descending order of the eigenvalues:

TABLE 2

| | | Eigenvectors | | | | | |
|---|---|---|---|---|---|---|---|
| index | Eigenvalues | CAN2 | RA(SCREW) | RV(SCREW) | CS | RV | SVC |
| 1 | 5.844 | −0.405 | −0.403 | −0.407 | −0.405 | −0.401 | −0.426 |
| 2 | 6.287 × 10⁻³ | −0.725 | −0.147 | −0.180 | −0.201 | 0.422 | 0.450 |
| 3 | 1.158 × 10⁻³ | −0.223 | 0.555 | −0.554 | 0.123 | −0.360 | 0.437 |
| 4 | 3.219 × 10⁻⁴ | −0.236 | 0.294 | −0.354 | 0.304 | 0.580 | −0.551 |
| 5 | 1.646 × 10⁻⁴ | 0.323 | −0.560 | −0.585 | 0.150 | 0.310 | 0.347 |
| 6 | 1.784 × 10⁻⁵ | 0.318 | 0.327 | −0.165 | −0.815 | 0.314 | 0.026 |

Each eigenvector is represented by a linear combination of the six signals sᵢ(t) and represents a basic mode of heart motion. An eigenvector's eigenvalue reflects the weight of that eigenvector and therefore the weight of the basic mode of motion represented by that eigenvector.

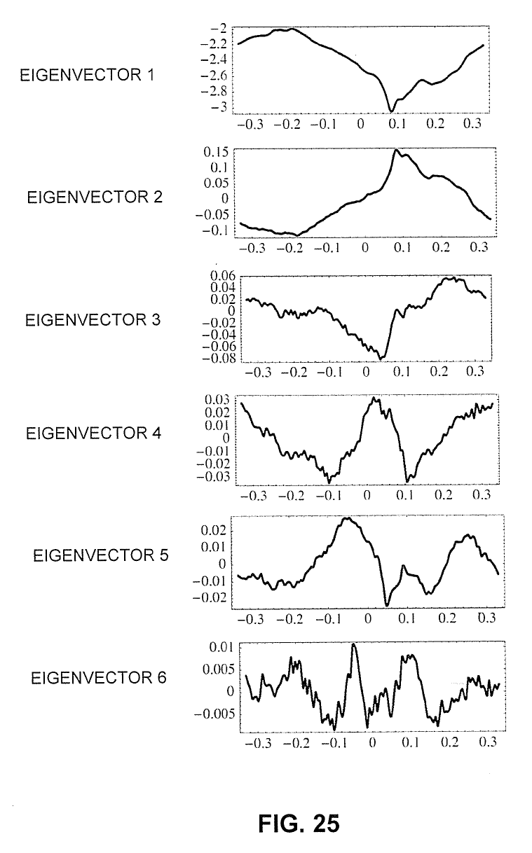

Accordingly, FIG. 25 presents the time-series plots for each eigenvector based on the linear combination of the six tomography signals as shown in TABLE 2.

By inspecting the absolute values of the coefficients associated with each tomography signal in the expression of an eigenvector, the weight carried by each tomography signal in a eigenvector is derived. As can be seen in TABLE 2, eigenvector 1 represents a common mode among all the electrodes, because each tomography signal carries approximately equal weight. Also apparent from TABLE 2 is that the common mode represented by eigenvector 1 is by far the most dominant mode of motion, because eigenvalue 1 is orders of magnitude larger than the rest.

For eigenvector 2, the main contributor is the tomography signal from CAN2, indicating that skin clip 2304 is measuring the interface impedance variation of can 2302 through which the AC voltage is driven. Also, since CAN2 is not located within the heart, the signal variations experienced by CAN2 is different from those experienced by other electrodes. These distinct signal variations on CAN2 are captured by eigenvector 2.

As to eigenvector 3, the two most dominant tomography signals come from RA(SCREW) and RV(SCREW). The two corresponding coefficients have opposite signs, indicating that electrodes 2306 and 2314 in FIG. 23 are moving in opposite directions. Such a movement represents a longitudinal contraction motion of the heart.

Following the same line of reasoning, for eigenvector 4, RV and SVC have coefficients of opposite signs, indicating a longitudinal contraction motion on the right side of the heart. As to eigenvector 5, RA(SCREW) and RV(SCREW) have coefficients of the same sign, whereas RV and SVC have coefficients of the opposite sign, indicating that the heart has a lateral contraction motion. For eigenvector 6, the dominant tomography signal is CS. The corresponding electrode is at the coronary sinus and does not move much.

As is evident from the above results and discussion, the subject invention provides numerous advantages. Advantages of various embodiments of the subject invention include, but are not limited to: low power consumption; real time discrimination of multiple lines of position possible (one or more); and noise tolerance, since the indicators are relative and mainly of interest in the time domain. A further advantage of this approach is that there is no need for additional catheters or electrodes for determining position. Rather the existing electrodes already used for pacing and defibrillation can be used to inject AC impulses at one or more frequencies designed not to interfere with the body or pacing apparatus. As such, the subject invention represents a significant contribution to the art.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method for evaluating movement of a tissue location in a subject, said method comprising:
(a) generating a continuous electric field so that said tissue location is present in said continuous field; and
(b) detecting a change in property of the continuous electric field at said tissue location to evaluate movement of said tissue location.

2. The method according to claim 1, wherein said evaluating comprises converting the detected change in property to a measurement of distance, location, or motion of the tissue location relative to a second location.

3. The method according to claim 2, wherein said movement is evaluated by calculating a motion between said tissue location and a second location.

4. The method according to claim 3, wherein said continuous field is generated from said second location.

5. The method according to claim 1, wherein said detecting comprises obtaining a signal from a sensing element stably associated with said tissue location, wherein said signal is induced in said sensing element by movement of said tissue location in said continuous field.

6. The method according to claim 1, wherein said detecting comprises determining a value for said property at least twice over a duration of time to evaluate movement of said tissue location.

7-9. (canceled)

10. The method according to claim 1, wherein said electric field is an oscillating electrical conduction current field.

11-15. (canceled)

16. The method according to claim 1, wherein said continuous electric field is generated between a source and at least one sensing element.

17. The method according to claim 1, wherein said continuous electric field is generated between a source and a ground, and said change in property is detecting by at least one sensing element that is not said ground.

18. The method according to claim 1, wherein said property is chosen from amplitude, phase and frequency.

19. The method according to claim 18, wherein said property is amplitude.

20. The method according to claim 19, wherein said detecting comprises detecting amplitude signals having the same phase and frequency.

21. The method according to claim 18, wherein said property is frequency.

22. The method according to claim 21, wherein said evaluating comprises determining velocity based on frequency.

23. The method according to claim 5, wherein said sensing element comprises at least one electrode.

24. The method according to claim 23, wherein said sensing element comprises two or more closely spaced electrodes.

25. The method according to claim 24, wherein said detecting comprises
(a) measuring a local gradient of the electric field between the closely spaced electrodes; and
(b) measuring a change in the value of the field.

26. The method according to claim 25, wherein said evaluating comprises calculating a location or motion of said tissue location based on both the measured gradient and the measured change of the value.

27. The method according to claim 1, wherein said tissue location is a cardiac location.

28. The method according to claim 27, wherein said cardiac location is a heart wall location.

29. The method according to claim 27, wherein said heart wall is a chamber wall or a ventricular wall.

30. The method according to claim 29, wherein said chamber wall is a septal wall.

31. The method according to claim 1, wherein said method is a method of determining timing of cardiac wall motion.

32. The method according to claim 31, wherein said method is a method of determining cardiac wall motion of a first cardiac wall relative to a second cardiac wall.

33. The method according to claim 32, wherein said method is a method of determining timing of cardiac wall motion of a first cardiac wall relative to a second cardiac wall.

34. The method according to claim 33, wherein said method is a method of detecting ventricular mechanical dyssynchrony.

35. The method according to claim 34, wherein said ventricular mechanical dyssynchrony is interventricular.

36. The method according to claim 34, wherein said ventricular mechanical dyssynchrony is intraventricular.

37. The method according to claim 34, wherein said method further comprises performing cardiac resynchronization therapy based on said detected dyssynchrony.

38. A system for evaluating movement of a tissue location, said system comprising:
(a) a continuous electric field generation element; and
(b) a continuous electric field sensing element configured to be stably associated with a tissue location; and
(c) a signal processing element configured to employ a signal obtained from said sensing element that is induced by movement of tissue location in said continuous electric field to evaluate movement of said tissue location.

39. A computer readable storage medium having a processing program stored thereon, wherein said processing program operates a processor operate a system according to claim 38 to perform a method according to claim 1.

40. A processor comprising a computer readable medium according to claim 39.

41. An adaptor device for modifying an implanted cardiac pacing device to be able to perform a method according to claim 1, said device comprising:
a processor according to claim 40; and
one or more adaptor elements for operably coupling to an implanted cardiac pacing device.

42. The adaptor device according to claim 41, wherein said adaptor device comprises at least one sensing element.

43. The adaptor device according to claim 42, wherein said sensing element is an electrode.

44. A kit comprising:
a computer readable storage medium according to claim 39.

45. The kit according to claim 44, wherein said computer readable storage medium is present in a processor according to claim 40.

46. The kit according to claim 45, wherein said processor is present in an adaptor device according to claim 41.

47. The kit according to claim 45, wherein said processor is present in a cardiac pacing device.

48. A device for evaluating movement of a cardiac location, said device comprising:
(a) a continuous electric field generation element; and
(b) a continuous electric field sensing element configured to be stably associated with said cardiac location; and
(c) a signal processing element configured to employ a signal obtained from said sensing element that is induced by movement of cardiac location in said continuous electric field to evaluate movement of said cardiac location.

49. The device according to claim 48, wherein said device further comprises a cardiac electrical stimulation element.

50. The device according to claim 49, wherein said device is a cardiac resynchronization therapy device.
